(19) United States
(12) Patent Application Publication
(10) Pub. No.: US 2022/0104445 A1
(43) Pub. Date: Apr. 7, 2022

(54) Crisphead Lettuce Varieties with Resistance to Bremia Lactucae

(71) Applicant: Bejo Zaden BV, Warmenhuizen (NL)

(72) Inventors: Ludovicus Pieter Anton Kamp, Breda (NL); Teunis Scheurwater, Nieuw Lekkerland (NL); Roelof Marinus Veenstra, Wieringerwaard (NL); Albertus Johannes Maria Schrijver, Warmenhuizen (NL)

(21) Appl. No.: 17/495,512

(22) Filed: Oct. 6, 2021

Related U.S. Application Data
(60) Provisional application No. 63/088,750, filed on Oct. 7, 2020.

(51) Int. Cl. A01H 6/14; A01H 5/12

(52) U.S. Cl. CPC A01H 6/1472; A01H 5/12

(57) ABSTRACT

Provided herein are various crisphead lettuce varieties, including seeds thereof and methods of using the varieties to generate additional lettuce varieties. The varieties are characterized by their color, size, shape, cold tolerance, and resistance to Bremia lactucae.

### CROSS-REFERENCE TO RELATED APPLICATIONS

The present application claims priority to U.S. Provisional Patent Application No. 63/088,750, filed Oct. 7, 2020, the content of which is incorporated by reference in its entirety.

### FIELD OF THE INVENTION

Disclosed herein are new varieties of crisphead lettuce.

### BACKGROUND OF THE INVENTION

Cultivated forms of lettuce are members of the highly polymorphic species Lactuca sativa, which is grown for its edible head and leaves. Lettuce is one of many members of the Asteraceae family. Other related plant species are, among many, Apium graveolens (celery), Foeniculum vulgare (fennel) and Cichorium intybus (chicory). As a crop, lettuce is grown commercially wherever environmental conditions permit the production of an economically viable yield.

A dicotyledonous species, L. sativa is known for its composite flowers made of individual florets. This composite flower is in fact a cluster of many small flowers, where some flowers have specialized, e.g., the “petals” are in fact ribbon-shaped flowers. Inside this area many small flowers or florets are present, each with an ovary, pappus, anthers, and a style with stigmas. The florets have five fused petals to form a corolla tube. The flowers mature from the outside toward the center, with the youngest flowers in the middle.

Lettuce was first cultivated in ancient Egypt where it was used for its leaves and oil containing seeds. From there, the crop spread to the Greeks and Romans. By 50 A.D., many types of lettuce were already described, and, as with many food crops, lettuce appeared in several mediaeval books, including herbal books. During the 16th through the 18th century, many new varieties were developed in Europe, and cultivars from the 18th century can still be found in gardens.

Lettuce cultivars (cultivated varieties) are available in a wide range of leaf colors, leaf shapes, and textures. In addition, lettuce is rich in vitamins (A, K) and is a good source of folate and iron. In dark green lettuce types, like Romaine, higher concentrations of β-carotene are present. The varied appearance, as well as the nutritional value of lettuce, contributes to the popularity of lettuce as a salad vegetable. Main lettuce types are: butterhead lettuce (round heads filled with thin, pliable leaves, and small, soft heads with an almost oily texture; L. sativa var. capitata), crisphead lettuce (round heads but thick, crisp leaves), which is divided further in Batavia (with open or closed, loosely filled heads; L. sativa var. capitata) and iceberg (with overlapping leaves forming a dense firm and closed head; L. sativa var. capitata), Romaine or cos lettuce (elongated upright leaves forming a loose, loaf-shaped head with dark green outer leaves; L. sativa var. longifolia), and leaf, cutting, or baby leaf lettuce (does not form a head; L. sativa var. crispy). Most cultivated lettuce types are diploid, where 2n=2x=18.

Lettuce is an open pollinated crop. Flowering plants self-pollinate because the floral structure (with a stylus growing through an anther cylinder) strongly promotes self-pollination. Lettuce is generally grown as a hardy annual; the crop is easily cultivated but requires relatively low temperatures to avoid premature flowering. Moreover, seeds from L. sativa are sensitive to high temperatures and soil salinity, both of which can affect germination.

Crisphead lettuce, including iceberg lettuce, is characterized by curved, overlapping leaves, and crispy, firm heads of a round shape. The overlapping outer leaves surround inner, white leaves, and the lettuce can have a sweet flavor. Crisphead lettuce can be sown in the fall or early spring, providing the opportunity for multiple seasons of produce. This type of lettuce is an important and valuable vegetable crop. Therefore, it is desirable to develop new varieties of crisphead lettuce having novel and exceptional traits.

### SUMMARY OF THE INVENTION

Provided herein is lettuce variety ‘1434365’, characterized by its color, size, and resistance to various isolates of Bremia lactucae. In one aspect, seed of lettuce variety ‘1434365’ is provided. Seed of variety ‘1434365’ has been deposited with NCIMB under Accession No. 43577.

Also provided herein are plants grown from seed of variety ‘1434365’, and plant parts and seeds produced by plants so grown.

Also provided herein are methods of generating offspring of variety ‘1434365’, including the steps of crossing a plant of variety ‘1434365’ with a second variety of lettuce plant. In some aspects, both plants are variety ‘1434365’. In other aspects, the second variety is selected for a desired trait which can be introduced into offspring, such that said offspring include physiological/morphological traits of variety ‘1434365’ and the desired trait(s) of the second variety.

Provided herein is lettuce variety ‘1434366’, characterized by its color, size, and resistance to various isolates of Bremia lactucae. In one aspect, seed of lettuce variety ‘1434366’ is provided. Seed of variety ‘1434366’ has been deposited with NCIMB under Accession No. 43576.

Also provided herein are plants grown from seed of variety ‘1434366’, and plant parts and seeds produced by plants so grown.

Also provided herein are methods of generating offspring of variety ‘1434366’, including the steps of crossing a plant of variety ‘1434366’ with a second variety of lettuce plant. In some aspects, both plants are variety ‘1434366’. In other aspects, the second variety is selected for a desired trait which can be introduced into offspring, such that said offspring include physiological/morphological traits of variety ‘1434366’ and the desired trait(s) of the second variety.

Provided herein is lettuce variety ‘1434367’, characterized by its color, size, and resistance to various isolates of Bremia lactucae. In one aspect, seed of lettuce variety ‘1434367’ is provided. Seed of variety ‘1434367’ has been deposited with NCIMB under Accession No. 43575.

Also provided herein are plants grown from seed of variety ‘1434367’, and plant parts and seeds produced by plants so grown.

Also provided herein are methods of generating offspring of variety ‘1434367’, including the steps of crossing a plant of variety ‘1434367’ with a second variety of lettuce plant. In some aspects, both plants are variety ‘1434367’. In other aspects, the second variety is selected for a desired trait which can be introduced into offspring, such that said offspring include physiological/morphological traits of variety ‘1434367’ and the desired trait(s) of the second variety.

Provided herein is lettuce variety ‘1434368’, characterized by its color, size, and resistance to various isolates of Bremia lactucae. In one aspect, seed of lettuce variety ‘1434368’ is provided. Seed of variety ‘1434368’ has been deposited with NCIMB under Accession No. 43574.

Also provided herein are plants grown from seed of variety ‘1434368’, and plant parts and seeds produced by plants so grown.

Also provided herein are methods of generating offspring of variety ‘1434368’, including the steps of crossing a plant of variety ‘1434368’ with a second variety of lettuce plant. In some aspects, both plants are variety ‘1434368’. In other aspects, the second variety is selected for a desired trait which can be introduced into offspring, such that said offspring include physiological/morphological traits of variety ‘1434368’ and the desired trait(s) of the second variety.

Provided herein is lettuce variety ‘1434369’, characterized by its color, size, and resistance to various isolates of Bremia lactucae. In one aspect, seed of lettuce variety ‘1434369’ is provided. Seed of variety ‘1434369’ has been deposited with NCIMB under Accession No. 43573.

Also provided herein are plants grown from seed of variety ‘1434369’, and plant parts and seeds produced by plants so grown.

Also provided herein are methods of generating offspring of variety ‘1434369’, including the steps of crossing a plant of variety ‘1434369’ with a second variety of lettuce plant. In some aspects, both plants are variety ‘1434369’. In other aspects, the second variety is selected for a desired trait which can be introduced into offspring, such that said offspring include physiological/morphological traits of variety ‘1434369’ and the desired trait(s) of the second variety.

Provided herein is lettuce variety ‘1434370’, characterized by its color, size, and resistance to various isolates of Bremia lactucae. In one aspect, seed of lettuce variety ‘1434370’ is provided. Seed of variety ‘1434370’ has been deposited with NCIMB under Accession No. 43571.

Also provided herein are plants grown from seed of variety ‘1434370’, and plant parts and seeds produced by plants so grown.

Also provided herein are methods of generating offspring of variety ‘1434370’, including the steps of crossing a plant of variety ‘1434370’ with a second variety of lettuce plant. In some aspects, both plants are variety ‘1434370’. In other aspects, the second variety is selected for a desired trait which can be introduced into offspring, such that said offspring include physiological/morphological traits of variety ‘1434370’ and the desired trait(s) of the second variety.

Provided herein is lettuce variety ‘1434371’, characterized by its color, size, and resistance to various isolates of Bremia lactucae. In one aspect, seed of lettuce variety ‘1434371’ is provided. Seed of variety ‘1434371’ has been deposited with NCIMB under Accession No. 43570.

Also provided herein are plants grown from seed of variety ‘1434371’, and plant parts and seeds produced by plants so grown.

Also provided herein are methods of generating offspring of variety ‘1434371’, including the steps of crossing a plant of variety ‘1434371’ with a second variety of lettuce plant. In some aspects, both plants are variety ‘1434371’. In other aspects, the second variety is selected for a desired trait which can be introduced into offspring, such that said offspring include physiological/morphological traits of variety ‘1434371’ and the desired trait(s) of the second variety.

Provided herein is lettuce variety ‘1434377’, characterized by its color, size, and resistance to various isolates of Bremia lactucae. In one aspect, seed of lettuce variety ‘1434377’ is provided. Seed of variety ‘1434377’ has been deposited with NCIMB under Accession No. 43569.

Also provided herein are plants grown from seed of variety ‘1434377’, and plant parts and seeds produced by plants so grown.

Also provided herein are methods of generating offspring of variety ‘1434377’, including the steps of crossing a plant of variety ‘1434377’ with a second variety of lettuce plant. In some aspects, both plants are variety ‘1434377’. In other aspects, the second variety is selected for a desired trait which can be introduced into offspring, such that said offspring include physiological/morphological traits of variety ‘1434377’ and the desired trait(s) of the second variety.

Provided herein is lettuce variety ‘P-2019107’, characterized by its color, size, and resistance to various isolates of Bremia lactucae. In one aspect, seed of lettuce variety ‘P-2019107’ is provided. Seed of variety ‘P-2019107’ has been deposited with NCIMB under Accession No. 43572.

Also provided herein are plants grown from seed of variety ‘P-2019107’, and plant parts and seeds produced by plants so grown.

Also provided herein are methods of generating offspring of variety ‘P-2019107’, including the steps of crossing a plant of variety ‘P-2019107’ with a second variety of lettuce plant. In some aspects, both plants are variety ‘P-2019107’. In other aspects, the second variety is selected for a desired trait which can be introduced into offspring, such that said offspring include physiological/morphological traits of variety ‘P-2019107’ and the desired trait(s) of the second variety.

### BRIEF DESCRIPTION OF THE DRAWINGS

FIG. 1 shows the breeding history of variety ‘1434365’;

FIG. 2 shows the breeding history of variety ‘1434366’;

FIG. 3 shows the breeding history of variety ‘1434367’;

FIG. 4 shows the breeding history of variety ‘1434368’;

FIG. 5 shows the breeding history of variety ‘1434369’;

FIG. 6 shows the breeding history of variety ‘1434370’;

FIG. 7 shows the breeding history of variety ‘1434371’;

FIG. 8 shows the breeding history of variety ‘1434377’; and

FIG. 9 shows the breeding history of variety ‘P-2019107’.

### DESCRIPTION OF THE INVENTION

In the description and tables which follow, a number of terms are used. To provide a clear and consistent understanding of the specification and claims, including the scope to be given such terms, the following definitions are provided:

Amplified Fragment Length Polymorphism (AFLP®): A PCR-based method of identifying polymorphisms through digestion with known restriction enzymes followed by visualization through use of radiography or fluorescence.

Allele: The allele is any of one or more alternative forms of a gene, all of which relate to one trait or characteristic. In a diploid cell or organism, the two alleles of a given gene occupy corresponding loci on a pair of homologous chromosomes.

Alternaria sonchi: A fungus of the phylum Ascomycota that causes Alternaria leaf spot in L. sativa.

Backcrossing: Backcrossing is a process in which a breeder repeatedly crosses hybrid progeny back to one of the parents, for example, a first generation hybrid F1 with one of the parental genotype of the F1 hybrid.

BC1: First backcross generation.

Bolting: The premature development of a flowering stalk, and subsequent seed

Bremia lactucae (Bl): A fungus of the class Oomycete that causes downy mildew in lettuce in cooler growing regions.

Cleaved Amplified Polymorphic Sequence (CAPS): A method of identifying genetic markers based on the length of restriction fragment lengths. Restriction fragments analyzed using CAPS are typically generated using a Restriction Fragment Length Polymorphism assay.

Core diameter: The diameter of the lettuce stem at the base of the cut head.

Core length: Length of the internal lettuce stem measured from the base of the cut and trimmed head to the tip of the stem.

Cotyledon: One of the first leaves of the embryo of a seed plant, typically one or more in monocotyledons, two in dicotyledons, and two or more in gymnosperms.

Clustered Regularly Interspaced Short Palindromic Repeats (CRISPR): Refers to DNA sequences found in bacteria that include portions of DNA introduced to the bacteria by a given virus. The concept is the basis of the CRISPR system of editing an organism's genome (see, e.g., U.S. Pat. No. 8,697,359, incorporated herein by reference in its entirety).

CRISPR-Associated System (CAS): A set of homologous genes, encoding enzymes, that reside in the genome near the site of CRISPR sequences. These enzymes target DNA sequences based on similarity to the viral sequences included in the CRISPR regions.

Embryo: A plant embryo is a portion of the seed including precursors of the leaves, stem, and root, and one or more cotyledons.

Erwinia carotavora: Bacteria of the class Gammaproteobacteria that can infect L. sativa.

F1, F2, F3 etc,: First, second, third, etc. filial generation of offspring of distinctly different parental types.

First water date: The date the seed first receives adequate moisture to germinate. This can and often does equal the planting date.

Frame diameter: The frame diameter is a measurement of the lettuce plant diameter at its widest point, measured from the outer most wrapper leaf tip to the outer most wrapper leaf tip.

Gene: As used herein, “gene” refers to a segment of nucleic acid. A gene can be introduced into a genome of a species, whether from a different species or from the same species, using transformation or various breeding methods.

Genetically-Modified Organism (GMO): An organism whose genome has been altered using some form of genetic engineering.

Green leaf lettuce: A type of lettuce characterized by having curled or incised leaves forming a loose green rosette that does not develop into a compact head.

Head diameter: Diameter of the cut and trimmed head, sliced vertically, and measured at the widest point perpendicular to the stem.

Head height: Height of the cut and trimmed head, sliced vertically, and measured from the base of the cut stem to the cap leaf.

Head weight: Weight of saleable lettuce head, cut and trimmed to market specifications.

Hypocotyl: The portion of the stem of an embryo plant beneath the cotyledons, but above the root.

Lettuce Big Vein virus (LBV): Big Vein is a disease of lettuce caused by Lettuce Mirafiori Big Vein Virus which is transmitted by the fungus Olpidium virulentus with vein clearing and leaf shrinkage resulting in plants of poor quality and reduced marketable value.

Lettuce Mosaic virus (LMV): A disease that can cause a stunted, deformed, or mottled pattern in young lettuce and yellow, twisted, and deformed leaves in older lettuce.

Marker-assisted recurrent backcrossing (MARB): A method of introducing a single locus of interest. The MARB method allows for maintenance of essential characteristics of the recurrent parent's genome. MARB is particularly effective for Quantitative Trait Loci (QTLs) that are highly variable.

Marker-assisted selection (MAS): A method of selecting a trait of interest based not on the trait, but on a marker associated with that trait.

Market stage: Market stage is the stage when a lettuce plant is ready for commercial lettuce harvest. In the case of an iceberg variety, the head is solid, and has reached an adequate size and weight.

Maturity date: Maturity refers to the stage when the plants are of full size or optimum weight, in marketable form or shape to be of commercial or economic value.

Meristematic cell: Cells of the meristem, which is a collection of undifferentiated cells in the plant. Meristematic cells include apical meristematic cells and lateral meristematic cells.

Nasonovia ribisnigri: A lettuce aphid that colonizes the innermost leaves of the lettuce plant, contaminating areas that cannot be treated easily with insecticides.

Pest: A bacterium, fungus, virus, insect, or animal that attacks or negatively affects a plant.

Plant: “Plant” includes plant cells, plant protoplasts, plant cells of tissue culture from which lettuce plants can be regenerated, plant calli, plant clumps, and plant cells that are intact in plants, or parts of plants such as pollen, flowers, seeds, leaves, stems and the like.

Promoter: A region of DNA upstream from the start of transcription and involved in recognition and binding of RNA polymerase and other proteins to initiate transcription. A “plant promoter” is a promoter capable of initiating transcription in plant cells.

Protoplast: A plant cell that has had its cell wall at least partially removed.

Pseudomonas cichorii: Bacteria of the phylum Proteobacteria that causes leaf blight and spotting in L. sativa.

Quantitative Trait Loci (QTL): Refers to genetic loci that control, to some degree, numerically representable traits that are usually continuously distributed.

Random Amplified Microsatellite Polymorphism (RAMP): A PCR-based method of identifying polymorphisms through use of simple sequence repeat markers and random amplified DNA polymorphism markers. RAMP is particularly useful for assessing genetic relationships in plant species.

Random Amplified DNA Polymorphism: A PCR-based method of amplifying random sections of DNA.

Ratio of head height/diameter: Head height divided by the head diameter is an indication of the head shape with <1 is flattened, 1=round, and >1 is pointed.

Recurrent Parent: The backcross parent, and member of an identifiable lineage or line that is improved by addition of a trait not found in that line.

Regeneration: Regeneration refers to the development of a plant from tissue culture.

Resistance: The character of a plant to restrict or even inhibit the development of a pest or pathogen in or on the plant and also the restriction of damage these organisms may cause in comparison to a susceptible variety and under comparable circumstances.

Restriction Fragment Length Polymorphism (RFLP): A marker in homologous DNA detectable based on fragments of different, specific lengths generated by known restriction enzymes.

Root tip: The terminal portion of the root of a plant.

Royal Horticultural Society of England (RHS): An organization that publishes an official botanical color chart quantitatively identifying colors according to a defined numbering system. The chart may be purchased from Royal Horticulture Society Enterprise Ltd., RHS Garden; Wisley, Woking; Surrey GU236QB, UK.

Romaine lettuce: A lettuce variety having elongated upright leaves forming a loose, loaf-shaped head and the outer leaves are usually dark green.

Simple sequence repeats (SSR): A DNA sequence having a number of motifs that are repeated.

Single nucleotide polymorphism (SNP): A common nucleotide variation at a given locus among a given population.

Tomato spotted wilt virus (TSWV): An RNA-containing virus of the genus Tospovirus that can infect L. sativa.

Transgenic: An organism that contains genetic material from an unrelated organism that has been artificially introduced.

Xanthomonas campestris: Bacteria of the phylum Proteobacteria that cause black rot in L. sativa.

### Lettuce Variety ‘1434365’

Provided herein is lettuce variety ‘1434365,’ a crisphead lettuce variety suitable for production in the summer and autumn harvesting seasons in, for example, Spain. Lettuce variety ‘1434365’ is the result of a cross between a mother crisphead line that was susceptible to Bremia lactucae and a Bremia lactucae-resistant crisphead line, and was selected following selfing and single-seed descent (SSD). These plants were chosen for their dark green leaf color and resistance to Bremia lactucae.

Lettuce variety ‘1434365’ has shown uniformity and stability for these traits with limited environmental influence on the traits. It has been self-pollinated through a sufficient number of generations with careful attention to uniformity of plant type. The variety has been propagated with continuous attention for uniformity.

### Breeding History

Crosses were made by spraying or misting water over the flowers, thereby inactivating pollen present on these flowers. This treatment enables the cross pollination of Lactuca plants.

F1 plants can be self-pollinated to produce the F2 generation, etc.

A Bremia lactucae-susceptible crisphead line was used as the mother variety, and was crossed with a Bremia lactucae-resistant line. The Bremia lactucae-resistant line is described in U.S. Pat. No. 10,575,479 and U.S. Patent Application Publication No. 2020/0029523, both of which are incorporated by reference herein in their entirety.

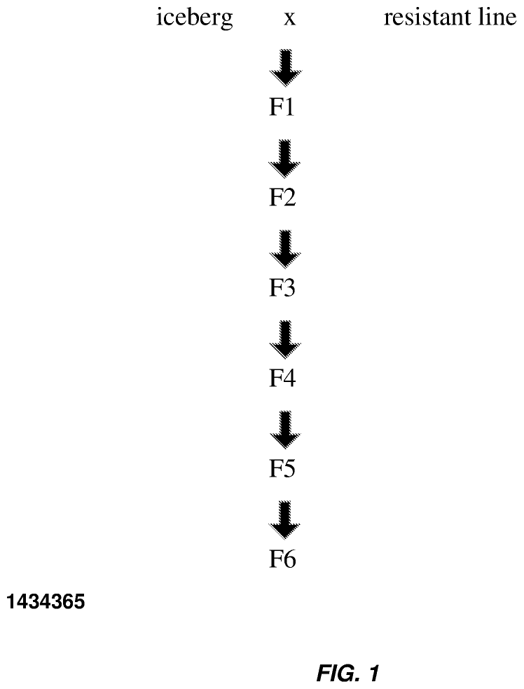

From these crosses, the F1 plants with the greatest resistance to Bremia lactucae were identified and a single plant was selected. Four lines were tested in the field for resistance to Bremia lactucae, and a single plant showing the greatest resistance was selected. Offspring of this best-performing line were selfed for two generations, followed by SSD of 50 lines, from which the best-performing line was selected (FIG. 1).

Seeds of all plants from this best-performing line were collectively harvested and designated crisphead variety ‘1434365’. A deposit of seeds of ‘1434365’ as disclosed above and recited in the appended claims has been made with the National Collections of Industrial Food and Marine Bacteria (NCIMB), Ferguson Building, Craibstone Estate, Bucksburn, Aberdeen AB21 9YA, United Kingdom. The date of the deposit was Feb. 7, 2020. All restrictions will be irrevocably removed upon granting of a patent, and the deposit is intended to meet all of the requirements of 37 C.F.R. § 1.801-1.809. The NCIMB Accession Number is 43577. The deposit will be maintained in the depository for a period of thirty years, or five years after the last request, or for the enforceable life of the patent, whichever is longer, and will be replaced as necessary during that period.

### Variety Description

Lettuce variety ‘1434365’ is a seed-propagated crisphead lettuce which is open pollinated. Variety ‘1434365’ is resistant to at least Bremia lactucae isolates Bl:16-36 and to Nasonovia ribisnigri:0. Lettuce variety ‘1434365’ has black seeds, exhibits dark green leaves and late to very late bolting under long day conditions, and is comparable to the variety ‘Asmara Rz’. With regard to this comparison, variety ‘1434365’ exhibits a different external structure with layers over layer while Asmara Rz in this regard has random characteristics. Variety ‘1434365’ has low ribs and the shape of ribs is flatround while ‘Asmara RZ’ has medium ribs and is ballround.

Characteristics of variety ‘1434365’ in concordance with the applicable UPOV Questionnaire, as compared to the ‘Asmara RZ’ variety, are provided below in Table 1.

TABLE 1

| | Characteristic | ‘1434365’ | | ‘Asmara RZ’ | |
|---|---|---|---|---|---|
| 1. | Seed: color | black | | black | |
| 2. | Plant: diameter (at market stage) | 55-60 | cm | 40-45 | cm |
| 3. | Plant: degree of overlapping of | strong | | strong | |
| | upper part of leaves | | | | |
| 4. | Leaf: number of leaves | not applicable | | not applicable | |
| 5. | Leaf: attitude | semi-erect | | semi-erect | |
| 6. | Leaf: number of divisions | absent or | | absent or | |
| | | very few | | very few | |
| 7. | Leaf: shape | broad obtrullate | | broad obtrullate | |
| 8. | Leaf: shape of apex | obcordate | | obcordate | |
| 9. | Leaf: longitudinal section | convex | | convex | |
| 10. | Leaf: width or lobes [only | not applicable | | not applicable | |
| | oakleaf type] | | | | |
| 11. | Leaf: anthocyanin coloration | absent or | | absent or | |
| | | very weak | | very weak | |
| 12. | Leaf: hue of anthocyanin | not applicable | | not applicable | |
| | coloration | | | | |
| 13. | Leaf: area covered by | very small | | very small | |
| | anthocyanin coloration | | | | |
| 14. | Leaf: color | green | | green | |
| 15. | Leaf: intensity of green color | dark | | dark | |
| 16. | Leaf: glossiness of upper side | medium | | medium | |
| 17. | Leaf: thickness | thick | | thick | |
| 18. | Leaf: blistering | absent or | | absent or | |
| | | very weak | | very weak | |
| 19. | Leaf: size of blisters | large | | medium | |
| 20. | Leaf: undulation of margin | Absent or | | Absent or | |
| | | very weak | | very weak | |
| 21. | Leaf: type of incisions of | irregularly | | irregularly | |
| | margin | dendate | | dendate | |
| 22. | Leaf: depth of incisions of | medium | | medium | |
| | margin | | | | |
| 23. | Leaf: depth of secondary | not applicable | | not applicable | |
| | incisions of margin | | | | |
| 24. | Leaf: density of incisions | sparse | | sparse | |
| | of margin | | | | |
| 25. | Leaf: venation | flabellate | | flabellate | |
| 26. | Head: size | Medium | | large | |
| 27. | Head: shape in longitudinal | Narrow oblate | | circular | |
| | section | | | | |
| 28. | Head: density | dense | | medium | |
| 29. | Stem: length | not applicable | | not applicable | |
| 30. | Stem: width | not applicable | | not applicable | |
| 31. | Stem: shape in longitudinal | not applicable | | not applicable | |
| | section | | | | |
| 32. | Stem: color | not applicable | | not applicable | |
| 33. | Stem: color of flesh | not applicable | | not applicable | |
| 34. | Time of harvest maturity | early | | early | |
| 35. | Time of beginning of bolting | late | | late | |
| 36. | Axillary sprouting | medium | | absent or weak | |
| 37. | Bolting stem: fasciation | absent or | | absent or | |
| | | very weak | | very weak | |
| 38. | Resistance to Bremia lactucae (Bl) | Present | | Present | |
| | Isolate Bl: 16EU | | | | |
| 39. | Resistance to Bremia lactucae (Bl) | Present | | Present | |
| | Isolate Bl: 17EU | | | | |
| 40. | Resistance to Bremia lactucae (Bl) | Present | | Present | |
| | Isolate Bl: 20EU | | | | |
| 41. | Resistance to Bremia lactucae (Bl) | Present | | Present | |
| | Isolate Bl: 21EU | | | | |
| 42. | Resistance to Bremia lactucae (Bl) | Present | | Present | |
| | Isolate Bl: 22EU | | | | |
| 43. | Resistance to Bremia lactucae (Bl) | Present | | Present | |
| | Isolate Bl: 23EU | | | | |
| 44. | Resistance to Bremia lactucae (Bl) | Present | | Present | |
| | Isolate Bl: 24EU | | | | |
| 45. | Resistance to Bremia lactucae (Bl) | Present | | Present | |
| | Isolate Bl: 25EU | | | | |
| 46. | Resistance to Bremia lactucae (Bl) | Present | | Present | |
| | Isolate Bl: 26EU | | | | |
| 47. | Resistance to Bremia lactucae (Bl) | Present | | Present | |
| | Isolate Bl: 27EU | | | | |
| 48. | Resistance to Bremia lactucae (Bl) | Present | | Present | |
| | Isolate Bl: 29EU | | | | |
| 49. | Resistance to Bremia lactucae (Bl) | Present | | Present | |
| | Isolate Bl: 30EU | | | | |
| 50. | Resistance to Bremia lactucae (Bl) | Present | | Present | |
| | Isolate Bl: 31EU | | | | |
| 51. | Resistance to Bremia lactucae (Bl) | Present | | Present | |
| | Isolate Bl: 33EU | | | | |
| 52. | Resistance to Bremia lactucae (Bl) | Present | | Present | |
| | Isolate Bl: 35EU | | | | |
| 53. | Resistance to Lettuce mosaic virus | Absent | | Absent | |
| | (LMV) Pathotype II | | | | |
| 54. | Resistance to Nasonovia ribisnigri | Present | | Absent | |
| | (Nr) | | | | |
| | Biotype Nr: 0 | | | | |
| 55. | Resistance to Fusarium oxysporum | Moderately | | susceptible | |
| | f. sp. lactucae (Fol) Race 1 | resistant | | | |
| 56. | Head: size class | 10/box | | 9-10/box | |
| 57. | Head: Shape | Flatround | | Balround | |
| 58. | Head: leaf wrap over the top | medium | | medium | |
| | of the head | | | | |
| 59. | Head: height head (mature stage; | 10.0 | cm | 11.0 | cm |
| | from cut till top) | | | | |
| 60. | Head: weight (mature stage; | 513 | gr | 720 | gr |
| | average of 4-5) | | | | |

| | | | |
|---|---|---|---|
| 61. | Resistance to Bremia lactucae (Bl) | Present | Present |
| | Isolate Bl: 32 EU | | |
| 62 | Resistance to Bremia lactucae (Bl) | Present | Present |
| | Isolate Bl: 34 EU | | |
| 63. | Resistance to Bremia lactucae (Bl) | Present | Present |
| | Isolate Bl: 36 EU | | |
| 64. | Resistance to Lettuce root aphid | Absent | Absent |
| | (Pb) | | |
| 65. | Resistance to Lettuce Necrotic | Absent | Absent |
| | Stunt Virus LNSV | | |
| 66. | Resistance to Lettuce Big Vein | Absent | Absent |
| | Virus LBW | | |
| 67. | Head: diameter of the cut of the | big | medium |
| | trunk | | |
| 68. | Head: height of the bottom ribs | low | medium |

### Lettuce Variety ‘1434366’

Provided herein is lettuce variety ‘1434366,’ a crisphead lettuce variety suitable for production in the summer and autumn harvesting seasons in, for example, Northern Italy. Lettuce variety ‘1434366’ is the result of a cross between a mother crisphead line that was susceptible to Bremia lactucae and a Bremia lactucae-resistant crisphead line, and was selected following a backcross, selfing, and SSD. These plants were chosen for their dark to very dark green leaf color and resistance to Bremia lactucae.

Lettuce variety ‘1434366’ has shown uniformity and stability for these traits, within the limits of environmental influence for the traits. It has been self-pollinated through a sufficient number of generations with careful attention to uniformity of plant type. The variety has been propagated with continuous attention for uniformity.

### Breeding History

Crosses were made by spraying or misting water over the flowers, thereby inactivating pollen present on these flowers. This treatment enables the cross pollination of Lactuca plants.

F1 plants can be self-pollinated to produce the F2 generation, etc.

A Bremia lactucae-susceptible crisphead line was used as the mother variety, and was crossed with a Bremia lactucae-resistant line. The Bremia lactucae-resistant line is described in U.S. Pat. No. 10,575,479 and U.S. Patent Application Publication No. 2020/0029523, both of which are incorporated by reference herein in their entirety.

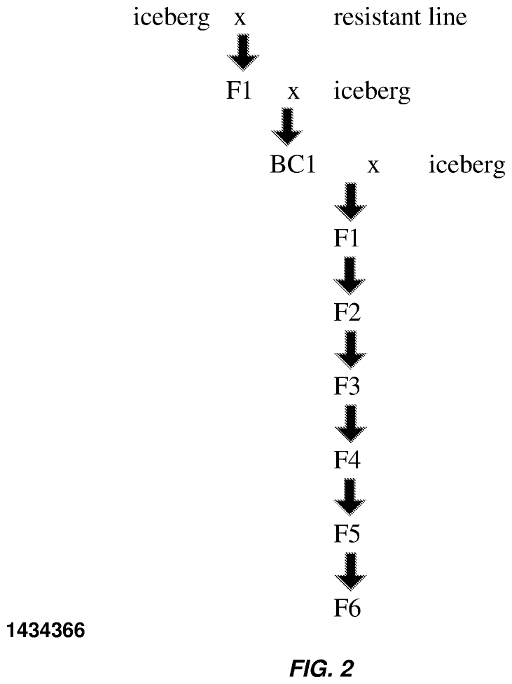

From these crosses, the F1 plants with the greatest resistance to Bremia lactucae were identified and a single plant was selected and backcrossed with the mother line. Following the backcross, 47 lines were tested (including in the field), and the best-performing plants were selected. From these best-performing lines, 20 SSD lines were made and the best-performing line following Bremia lactucae resistance testing was selected. Four lines were tested in the field for resistance to Bremia lactucae, and a single plant showing the greatest resistance was selected. The best-performing plant was selfed for five generations at different times in the field, and the best line was selected (FIG. 2).

Seeds of all plants from this best-performing line were collectively harvested and designated crisphead variety ‘1434366’. A deposit of seeds of ‘1434366’ as disclosed above and recited in the appended claims has been made with the National Collections of Industrial, Food and Marine Bacteria (NCIMB), Ferguson Building, Craibstone Estate, Bucksburn, Aberdeen AB21 9YA, United Kingdom. The date of the deposit was Feb. 7, 2020. All restrictions will be irrevocably removed upon granting of a patent, and the deposit is intended to meet all of the requirements of 37 C.F.R. § 1.801-1.809. The NCIMB Accession Number is 43576. The deposit will be maintained in the depository for a period of thirty years, or five years after the last request, or for the enforceable life of the patent, whichever is longer, and will be replaced as necessary during that period.

### Variety Description

Lettuce variety ‘1434366’ is a seed-propagated crisphead lettuce which is open pollinated. Variety ‘1434366’ is resistant to at least Bremia lactucae isolates Bl:16-36 and to Nasonovia ribisnigri:0. Lettuce variety ‘1434366’ has black seeds, exhibits dark to very dark green leaves and late to very late bolting under long day conditions, and is comparable to the variety ‘Brice’. With regard to this comparison, variety ‘1434366’ exhibits a darker leaf color, rounder and longer spikes at the leaf edge, and is smaller than ‘Brice’.

Characteristics of ‘1434366’ in concordance with the applicable UPOV Questionnaire, as compared to the ‘Brice’ variety, are provided below in Table 2.

TABLE 2

| | Characteristic | ‘1434366’ | | ‘Brice’ | |
|---|---|---|---|---|---|
| 1. | Seed: color | black | | black | |
| 2. | Plant: diameter (at market stage) | 40-45 | cm | 45-50 | cm |
| 3. | Plant: degree of overlapping | strong | | strong | |
| | of upper part of leaves | | | | |
| 4. | Leaf: number of leaves | not applicable | | not applicable | |
| 5. | Leaf: attitude | semi-erect | | semi-erect | |
| 6. | Leaf: number of divisions | absent or | | absent or | |
| | | very few | | very few | |
| 7. | Leaf: shape | broad | | broad | |
| | | obtrullate | | obtrullate | |
| 8. | Leaf: shape of apex | obcordate | | obcordate | |
| 9. | Leaf: longitudinal section | convex | | convex | |
| 10. | Leaf: width or lobes [only | not applicable | | not applicable | |
| | oakleaf type] | | | | |
| 11. | Leaf: anthocyanin coloration | absent or | | absent or | |
| | | very weak | | very weak | |
| 12. | Leaf: hue of anthocyanin | not applicable | | not applicable | |
| | coloration | | | | |
| 13. | Leaf: area covered by anthocyanin | very small | | very small | |
| | coloration | | | | |
| 14. | Leaf: color | green | | green | |
| 15. | Leaf: intensity of green color | very dark | | very dark | |
| 16. | Leaf: glossiness of upper side | medium | | medium | |
| 17. | Leaf: thickness | thick | | thick | |
| 18. | Leaf: blistering | absent or | | absent or | |
| | | very weak | | very weak | |
| 19. | Leaf: size of blisters | large | | medium | |
| 20. | Leaf: undulation of margin | Absent or | | Absent or | |
| | | very weak | | very weak | |
| 21. | Leaf: type of incisions of margin | bidendate | | irregularly | |
| | | | | dendate | |
| 22. | Leaf: depth of incisions of margin | deep | | medium | |
| 23. | Leaf: depth of secondary incisions | shallow | | not applicable | |
| | of margin | | | | |
| 24. | Leaf: density of incisions of | sparse | | sparse | |
| | margin | | | | |
| 25. | Leaf: venation | flabellate | | flabellate | |
| 26. | Head: size | medium | | large | |
| 27. | Head: shape in longitudinal section | circular | | circular | |
| 28. | Head: density | dense | | medium | |
| 29. | Stem: length | not applicable | | not applicable | |
| 30. | Stem: width | not applicable | | not applicable | |
| 31. | Stem: shape in longitudinal section | not applicable | | not applicable | |
| 32. | Stem: color | not applicable | | not applicable | |
| 33. | Stem: color of flesh | not applicable | | not applicable | |
| 34. | Time of harvest maturity | late | | medium | |
| 35. | Time of beginning of bolting | very late | | late | |
| 36. | Axillary sprouting | medium | | absent or weak | |
| 37. | Bolting stem: fasciation | absent or | | absent or | |
| | | very weak | | very weak | |
| 38. | Resistance to Bremia lactucae (Bl) | Present | | Present | |
| | Isolate Bl: 16EU | | | | |
| 39. | Resistance to Bremia lactucae (Bl) | Present | | Present | |
| | Isolate Bl: 17EU | | | | |
| 40. | Resistance to Bremia lactucae (Bl) | Present | | Present | |
| | Isolate Bl: 20EU | | | | |
| 41. | Resistance to Bremia lactucae (Bl) | Present | | Present | |
| | Isolate Bl: 21EU | | | | |
| 42. | Resistance to Bremia lactucae (Bl) | Present | | Present | |
| | Isolate Bl: 22EU | | | | |
| 43. | Resistance to Bremia lactucae (Bl) | Present | | Present | |
| | Isolate Bl: 23EU | | | | |
| 44. | Resistance to Bremia lactucae (Bl) | Present | | Present | |
| | Isolate Bl: 24EU | | | | |
| 45. | Resistance to Bremia lactucae (Bl) | Present | | Present | |
| | Isolate Bl: 25EU | | | | |
| 46. | Resistance to Bremia lactucae (Bl) | Present | | Present | |
| | Isolate Bl: 26EU | | | | |
| 47. | Resistance to Bremia lactucae (Bl) | Present | | Present | |
| | Isolate Bl: 27EU | | | | |
| 48. | Resistance to Bremia lactucae (Bl) | Present | | Present | |
| | Isolate Bl: 29EU | | | | |
| 49. | Resistance to Bremia lactucae (Bl) | Present | | Present | |
| | Isolate Bl: 30EU | | | | |
| 50. | Resistance to Bremia lactucae (Bl) | Present | | Present | |
| | Isolate Bl: 31EU | | | | |
| 51. | Resistance to Bremia lactucae (Bl) | Present | | Present | |
| | Isolate Bl: 33EU | | | | |
| 52. | Resistance to Bremia lactucae (Bl) | Present | | Present | |
| | Isolate Bl: 35EU | | | | |
| 53. | Resistance to Lettuce mosaic virus | Absent | | Absent | |
| | (LMV) Pathotype II | | | | |
| 54. | Resistance to Nasonovia ribisnigri | Present | | Present | |
| | (Nr) | | | | |
| | Biotype Nr: 0 | | | | |
| 55. | Resistance to Fusarium oxysporum | Moderately | | susceptible | |
| | f. sp. lactucae (Fol) Race 1 | resistant | | | |
| 56. | Head: size class | 10/box | | 9-10/box | |
| 57. | Head: Shape | balround | | balround | |
| 58. | Head: leaf wrap over the top of the | medium | | medium | |
| | head | | | | |
| 59. | Head: height head (mature stage; | 13.0 | cm | 10.0 | cm |
| | from cut till top) | | | | |
| 60. | Head: weight (mature stage; | 698 | gr | 570 | gr |
| | average of 4-5) | | | | |

| | | | |
|---|---|---|---|
| 61. | Resistance to Bremia lactucae (Bl) | Present | Present |
| | Isolate Bl: 32 EU | | |
| 62 | Resistance to Bremia lactucae (Bl) | Present | Present |
| | Isolate Bl: 34 EU | | |
| 63. | Resistance to Bremia lactucae (Bl) | Present | Present |
| | Isolate Bl: 36 EU | | |
| 64. | Resistance to Lettuce root aphid | Absent | Absent |
| | (Pb) | | |
| 65. | Resistance to Lettuce Necrotic | Absent | Absent |
| | Stunt Virus LNSV | | |
| 66. | Resistance to Lettuce Big Vein | Absent | Absent |
| | Virus LBVV | | |
| 67. | Head: diameter of the cut of the | Big | Medium |
| | trunk | | |
| 68. | Head: height of the bottom ribs | medium | medium |

### Lettuce Variety ‘1434367’

Provided herein is lettuce variety ‘1434367,’ a crisphead lettuce variety suitable for production in the autumn and winter harvesting seasons in, for example, Spain. Lettuce variety ‘1434367’ is the result of a cross between a mother crisphead line that was susceptible to Bremia lactucae and a Bremia lactucae-resistant crisphead line, and was selected following backcrosses and selfing. These plants were chosen for their dark green leaf color and resistance to Bremia lactucae.

Lettuce variety ‘1434367’ has uniformity and stability for these traits, within the limits of environmental influence for the traits. It has been self-pollinated through a sufficient number of generations with careful attention to uniformity of plant type. The variety has been propagated with continuous attention for uniformity.

### Breeding History

Crosses were made by spraying or misting water over the flowers, thereby inactivating pollen present on these flowers. This treatment enables the cross pollination of Lactuca plants.

F1 plants can be self-pollinated to produce the F2 generation, etc.

A Bremia lactucae-susceptible crisphead line was used as the mother variety, and was crossed with a Bremia lactucae-resistant line. The Bremia lactucae-resistant line is described in U.S. Pat. No. 10,575,479 and U.S. Patent Application Publication No. 2020/0029523, both of which are incorporated by reference herein in their entirety.

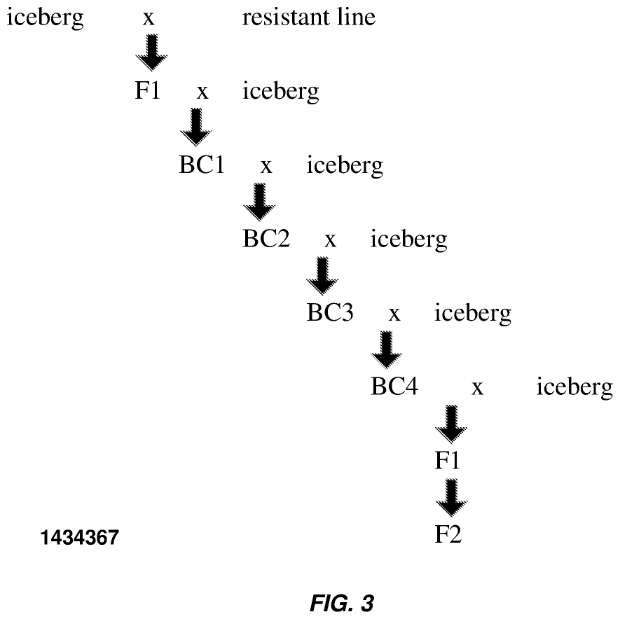

From these crosses, the F1 plants with the greatest resistance to Bremia lactucae were identified and a single plant was selected and backcrossed with the mother line. This procedure was repeated three additional times (backcrossing of best-performing offspring line with the original mother line). Following the backcrosses, the last, best-performing generation was selfed for two generations. The most uniform line was then selected (FIG. 3).

Seeds of all plants from this most-uniform line were collectively harvested and designated crisphead variety ‘1434367’. A deposit of seeds of ‘1434367’ as disclosed above and recited in the appended claims has been made with the National Collections of Industrial, Food and Marine Bacteria (NCIMB), Ferguson Building, Craibstone Estate, Bucksburn, Aberdeen AB21 9YA, United Kingdom. The date of the deposit was Feb. 7, 2020. All restrictions will be irrevocably removed upon granting of a patent, and the deposit is intended to meet all of the requirements of 37 C.F.R. § 1.801-1.809. The NUMB Accession Number is 43575. The deposit will be maintained in the depository for a period of thirty years, or five years after the last request, or for the enforceable life of the patent, whichever is longer, and will be replaced as necessary during that period.

### Variety Description

Lettuce variety ‘1434367’ is a seed-propagated crisphead lettuce which is open pollinated. Variety ‘1434367’ is resistant to at least Bremia lactucae isolates Bl:16-36 and to Nasonovia ribisnigri:0. Lettuce variety ‘1434367’ has black seeds, exhibits dark green leaves and late bolting under long day conditions, and is comparable to the variety ‘Damice’. With regard to this comparison, variety ‘1434367’ is larger than ‘Damice,’ and is resistant to Nasonovia ribisnigri:0, whereas ‘Damice’ is not.

Characteristics of ‘1434367’ in concordance with the applicable UPOV Questionnaire, as compared to the ‘Damice’ variety, are provided below in Table 3.

TABLE 3

| | Characteristic | ‘1434367’ | | ‘Damice’ | |
|---|---|---|---|---|---|
| 1. | Seed: color | black | | black | |
| 2. | Plant: diameter (at market stage) | 45-50 | cm | 40-45 | cm |
| 3. | Plant: degree of overlapping of | strong | | strong | |
| | upper part of leaves | | | | |
| 4. | Leaf: number of leaves | not applicable | | not applicable | |
| 5. | Leaf: attitude | semi-erect | | semi-erect | |
| 6. | Leaf: number of divisions | absent or | | absent or | |
| | | very few | | very few | |
| 7. | Leaf: shape | broad | | broad | |
| | | obtrullate | | obtrullate | |
| 8. | Leaf: shape of apex | obcordate | | obcordate | |
| 9. | Leaf: longitudinal section | convex | | convex | |
| 10. | Leaf: width or lobes [only | not applicable | | not applicable | |
| | oakleaf type] | | | | |
| 11. | Leaf: anthocyanin coloration | absent or | | absent or | |
| | | very weak | | very weak | |
| 12. | Leaf: hue of anthocyanin | not | | not | |
| | coloration | applicable | | applicable | |
| 13. | Leaf: area covered by anthocyanin | very small | | very small | |
| | coloration | | | | |
| 14. | Leaf: color | green | | green | |
| 15. | Leaf: intensity of green color | dark | | dark | |
| 16. | Leaf: glossiness of upper side | medium | | medium | |
| 17. | Leaf: thickness | thick | | thick | |
| 18. | Leaf: blistering | weak | | medium | |
| 19. | Leaf: size of blisters | medium | | medium | |
| 20. | Leaf: undulation of margin | weak | | weak | |
| 21. | Leaf: type of incisions of margin | irregularly | | irregularly | |
| | | dendate | | dendate | |
| 22. | Leaf: depth of incisions of margin | shallow | | shallow | |
| 23. | Leaf: depth of secondary incisions | not | | not | |
| | of margin | applicable | | applicable | |
| 24. | Leaf: density of incisions of | sparse | | sparse | |
| | margin | | | | |
| 25. | Leaf: venation | flabellate | | flabellate | |
| 26. | Head: size | large | | large | |
| 27. | Head: shape in longitudinal section | narrow oblate | | narrow oblate | |
| 28. | Head: density | medium | | dense | |
| 29. | Stem: length | not applicable | | not applicable | |
| 30. | Stem: width | not applicable | | not applicable | |
| 31. | Stem: shape in longitudinal section | not applicable | | not applicable | |
| 32. | Stem: color | not applicable | | not applicable | |
| 33. | Stem: color of flesh | not applicable | | not applicable | |
| 34. | Time of harvest maturity | early | | early | |
| 35. | Time of beginning of bolting | medium | | medium | |
| 36. | Axillary sprouting | absent or weak | | medium | |
| 37. | Bolting stem: fasciation | absent or | | absent or | |
| | | very weak | | very weak | |
| 38. | Resistance to Bremia lactucae (Bl) | Present | | Present | |
| | Isolate Bl: 16EU | | | | |
| 39. | Resistance to Bremia lactucae (Bl) | Present | | Present | |
| | Isolate Bl: 17EU | | | | |
| 40. | Resistance to Bremia lactucae (Bl) | Present | | Present | |
| | Isolate Bl: 20EU | | | | |
| 41. | Resistance to Bremia lactucae (Bl) | Present | | Present | |
| | Isolate Bl: 21EU | | | | |
| 42. | Resistance to Bremia lactucae (Bl) | Present | | Present | |
| | Isolate Bl: 22EU | | | | |
| 43. | Resistance to Bremia lactucae (Bl) | Present | | Present | |
| | Isolate Bl: 23EU | | | | |
| 44. | Resistance to Bremia lactucae (Bl) | Present | | Present | |
| | Isolate Bl: 24EU | | | | |
| 45. | Resistance to Bremia lactucae (Bl) | Present | | Present | |
| | Isolate Bl: 25EU | | | | |
| 46. | Resistance to Bremia lactucae (Bl) | Present | | Present | |
| | Isolate Bl: 26EU | | | | |
| 47. | Resistance to Bremia lactucae (Bl) | Present | | Present | |
| | Isolate Bl: 27EU | | | | |
| 48. | Resistance to Bremia lactucae (Bl) | Present | | Present | |
| | Isolate Bl: 29EU | | | | |
| 49. | Resistance to Bremia lactucae (Bl) | Present | | Present | |
| | Isolate Bl: 30EU | | | | |
| 50. | Resistance to Bremia lactucae (Bl) | Present | | Present | |
| | Isolate Bl: 31EU | | | | |
| 51. | Resistance to Bremia lactucae (Bl) | Present | | Present | |
| | Isolate Bl: 33EU | | | | |
| 52. | Resistance to Bremia lactucae (Bl) | Present | | Present | |
| | Isolate Bl: 35EU | | | | |
| 53. | Resistance to Lettuce mosaic virus | Absent | | Absent | |
| | (LMV) Pathotype II | | | | |
| 54. | Resistance to Nasonovia ribisnigri | Present | | Absent | |
| | (Nr) | | | | |
| | Biotype Nr: 0 | | | | |
| 55. | Resistance to Fusarium oxysporum | susceptible | | susceptible | |
| | f. sp. lactucae (Fol) Race 1 | | | | |
| 56. | Head: size class | 8-9/box | | 9-10/box | |
| 57. | Head: Shape | Little flat | | ball round | |
| | | round | | | |
| 58. | Head: leaf wrap over the top of the | good | | good | |
| | head | | | | |
| 59. | Head: height head (mature stage; | 14.0 | cm | 13.0 | cm |
| | from cut till top) | | | | |
| 60. | Head: weight (mature stage; | 843 | gr | 898 | gr |
| | average of 4-5) | | | | |

| | | | |
|---|---|---|---|
| 61. | Resistance to Bremia lactucae (Bl) | Present | Present |
| | Isolate Bl: 32 EU | | |
| 62 | Resistance to Bremia lactucae (Bl) | Present | Present |
| | Isolate Bl: 34 EU | | |
| 63. | Resistance to Bremia lactucae (Bl) | Present | Present |
| | Isolate Bl: 36 EU | | |
| 64. | Resistance to Lettuce root aphid | Absent | Absent |
| | (Pb) | | |
| 65. | Resistance to Lettuce Necrotic | Absent | Absent |
| | Stunt Virus LNSV | | |
| 66. | Resistance to Lettuce Big Vein | Absent | Absent |
| | Virus LBVV | | |
| 67. | Head: diameter of the cut of the | medium | big |
| | trunk | | |
| 68. | Head: height of the bottom ribs | high | medium |

### Lettuce Variety ‘1434368’

Provided herein is lettuce variety ‘1434368,’ a crisphead lettuce variety suitable for production in the spring and autumn harvesting seasons in, for example, Northern Europe. Lettuce variety ‘1434368’ is the result of a cross between a mother crisphead line that was susceptible to Bremia lactucae and a Bremia lactucae-resistant crisphead line, and was selected following backcrosses and selfing. These plants were chosen for their dark to very dark green leaf color and resistance to Bremia lactucae.

Lettuce variety ‘1434368’ has uniformity and stability for these traits, within the limits of environmental influence for the traits. It has been self-pollinated through a sufficient number of generations with careful attention to uniformity of plant type. The variety has been propagated with continuous attention for uniformity.

### Breeding History

Crosses were made by spraying or misting water over the flowers, thereby inactivating pollen present on these flowers. This treatment enables the cross pollination of Lactuca plants.

F1 plants can be self-pollinated to produce the F2 generation, etc.

A Bremia lactucae-susceptible crisphead line was used as the mother variety, and was crossed with a Bremia lactucae-resistant line. The Bremia lactucae-resistant line is described in U.S. Pat. No. 10,575,479 and U.S. Patent Application Publication No. 2020/0029523, both of which are incorporated by reference herein in their entirety.

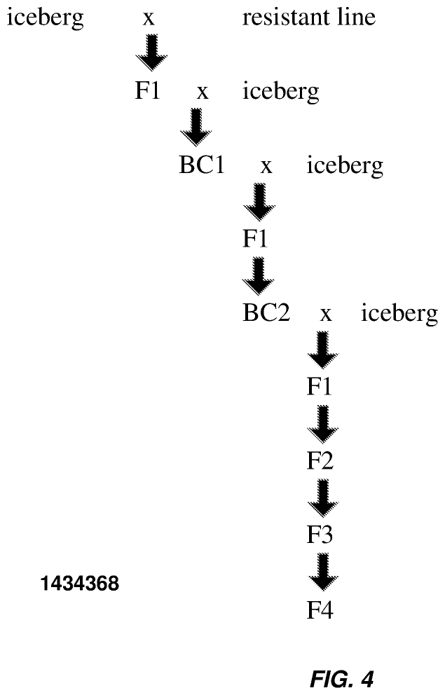

From these crosses, the F1 plants with the greatest resistance to Bremia lactucae were identified and a single plant was selected and backcrossed with the mother line. This procedure was repeated one more time (backcrossing of best-performing offspring line with the original mother line). Following the backcrosses, the last, best-performing generation was selfed for four generations. The best-performing line was then selected (FIG. 4).

Seeds of all plants from this most-uniform line were collectively harvested and designated crisphead variety ‘1434368’. A deposit of seeds of ‘1434368’ as disclosed above and recited in the appended claims has been made with the National Collections of Industrial, Food and Marine Bacteria (NCIMB), Ferguson Building, Craibstone Estate, Bucksburn, Aberdeen AB21 9YA, United Kingdom. The date of the deposit, was Feb. 7, 2020. All restrictions will be irrevocably removed upon granting of a patent, and the deposit is intended to meet all of the requirements of 37 C.F.R. § 1.801-1.809. The NCIMB Accession Number is 43574. The deposit will be maintained in the depository for a period of thirty years, or five years after the last request, or for the enforceable life of the patent, whichever is longer, and will be replaced as necessary during that period.

### Variety Description

Lettuce variety ‘1434368’ is a seed-propagated crisphead lettuce which is open pollinated. Variety ‘1434368’ is resistant to at least Bremia lactucae isolates Bl:16-36, Nasonovia ribisnigri:0, and LMV Strain LS-1. Lettuce variety ‘1434368’ has black seeds, exhibits dark to very dark green leaves and late bolting under long day conditions, and is comparable to the variety ‘Salinas’. With regard to this comparison, variety ‘1434368’ exhibits slower bolting and a wrap of 50% over the head (compared to 80% for ‘Salinas’).

Characteristics of ‘1434368’ in concordance with the applicable UPOV Questionnaire, as compared to the ‘Salinas’ variety, are provided below in Table 4.

TABLE 4

| | Characteristic | ‘1434368’ | | ‘Salinas’ | |
|---|---|---|---|---|---|
| 1. | Seed: color | black | | black | |
| 2. | Plant: diameter (at market stage) | 35-40 | cm | 35-40 | cm |
| 3. | Plant: degree of overlapping of | strong | | strong | |
| | upper part of leaves | | | | |
| 4. | Leaf: number of leaves | not applicable | | not applicable | |
| 5. | Leaf: attitude | semi-erect | | semi-erect | |
| 6. | Leaf: number of divisions | absent or | | absent or | |
| | | very few | | very few | |
| 7. | Leaf: shape | medium | | broad | |
| | | oblate | | obtrullate | |
| 8. | Leaf: shape of apex | obcordate | | obcordate | |
| 9. | Leaf: longitudinal section | convex | | convex | |
| 10. | Leaf: width or lobes [only | not applicable | | not applicable | |
| | oakleaf type] | | | | |
| 11. | Leaf: anthocyanin coloration | absent or | | absent or | |
| | | very weak | | very weak | |
| 12. | Leaf: hue of anthocyanin | not applicable | | not applicable | |
| | coloration | | | | |
| 13. | Leaf: area covered by | very small | | very small | |
| | anthocyanin coloration | | | | |
| 14. | Leaf: color | green | | green | |
| 15. | Leaf: intensity of green color | very dark | | very dark | |
| 16. | Leaf: glossiness of upper side | medium | | strong | |
| 17. | Leaf: thickness | thick | | thick | |
| 18. | Leaf: blistering | absent or | | absent or | |
| | | very weak | | very weak | |
| 19. | Leaf: size of blisters | large | | large | |
| 20. | Leaf: undulation of margin | weak | | weak | |
| 21. | Leaf: type of incisions of | regularly | | irregularly | |
| | margin | dendate | | dendate | |
| 22. | Leaf: depth of incisions of | absent or | | absent or | |
| | margin | very shallow | | very shallow | |
| 23. | Leaf: depth of secondary | not applicable | | not applicable | |
| | incisions of margin | | | | |
| 24. | Leaf: density of incisions of | very sparse | | very sparse | |
| | margin | | | | |
| 25. | Leaf: venation | flabellate | | flabellate | |
| 26. | Head: size | large | | large | |
| 27. | Head: shape in longitudinal | Narrow oblate | | circular | |
| | section | | | | |
| 28. | Head: density | medium | | dense | |
| 29. | Stem: length | not applicable | | not applicable | |
| 30. | Stem: width | not applicable | | not applicable | |
| 31. | Stem: shape in longitudinal | not applicable | | not applicable | |
| | section | | | | |
| 32. | Stem: color | not applicable | | not applicable | |
| 33. | Stem: color of flesh | not applicable | | not applicable | |
| 34. | Time of harvest maturity | medium | | medium | |
| 35. | Time of beginning of bolting | late | | late | |
| 36. | Axillary sprouting | absent or weak | | medium | |
| 37. | Bolting stem: fasciation | absent or weak | | absent or weak | |
| 38. | Resistance to Bremia lactucae | Present | | Absent | |
| | (Bl) | | | | |
| | Isolate Bl: 16EU | | | | |
| 39. | Resistance to Bremia lactucae | Present | | Absent | |
| | (Bl) | | | | |
| | Isolate Bl: 17EU | | | | |
| 40. | Resistance to Bremia lactucae | Present | | Absent | |
| | (Bl) | | | | |
| | Isolate Bl: 20EU | | | | |
| 41. | Resistance to Bremia lactucae | Present | | Absent | |
| | (Bl) | | | | |
| | Isolate Bl: 21EU | | | | |
| 42. | Resistance to Bremia lactucae | Present | | Absent | |
| | (Bl) | | | | |
| | Isolate Bl: 22EU | | | | |
| 43. | Resistance to Bremia lactucae | Present | | Absent | |
| | (Bl) | | | | |
| | Isolate Bl: 23EU | | | | |
| 44. | Resistance to Bremia lactucae | Present | | Absent | |
| | (Bl) | | | | |
| | Isolate Bl: 24EU | | | | |
| 45. | Resistance to Bremia lactucae | Present | | Absent | |
| | (Bl) | | | | |
| | Isolate Bl: 25EU | | | | |
| 46. | Resistance to Bremia lactucae (Bl) | Present | | Absent | |
| | Isolate Bl: 26EU | | | | |
| 47. | Resistance to Bremia lactucae (Bl) | Present | | Absent | |
| | Isolate Bl: 27EU | | | | |
| 48. | Resistance to Bremia lactucae (Bl) | Present | | Absent | |
| | Isolate Bl: 29EU | | | | |
| 49. | Resistance to Bremia lactucae (Bl) | Present | | Absent | |
| | Isolate Bl: 30EU | | | | |
| 50. | Resistance to Bremia lactucae (Bl) | Present | | Absent | |
| | Isolate Bl: 31EU | | | | |
| 51. | Resistance to Bremia lactucae (Bl) | Present | | Absent | |
| | Isolate Bl: 33EU | | | | |
| 52. | Resistance to Bremia lactucae (Bl) | Present | | Absent | |
| | Isolate Bl: 35EU | | | | |
| 53. | Resistance to Lettuce mosaic | Present | | Absent | |
| | virus (LMV) Pathotype II | | | | |
| 54. | Resistance to Nasonovia ribisnigri | Present | | Absent | |
| | (Nr) | | | | |
| | Biotype Nr: 0 | | | | |
| 55. | Resistance to Fusarium | susceptible | | Moderately | |
| | oxysporum f. sp. lactucae (Fol) | | | resistant | |
| | Race 1 | | | | |
| 56. | Head: size class | 9-10/box | | 8-10/box | |
| 57. | Head: Shape | balround | | balround | |
| 58. | Head: leaf wrap over the top of | medium | | medium | |
| | the head | | | | |
| 59. | Head: height head (mature stage; | 13.0 | cm | 12.0 | cm |
| | from cut till top) | | | | |
| 60. | Head: weight (mature stage; | 692 | gr | 875 | gr |
| | average of 4-5) | | | | |

| | | | |
|---|---|---|---|
| 61. | Resistance to Bremia lactucae (Bl) | present | absent |
| | Isolate Bl: 32 EU | | |
| 62 | Resistance to Bremia lactucae (Bl) | present | absent |
| | Isolate Bl: 34 EU | | |
| 63. | Resistance to Bremia lactucae (Bl) | present | absent |
| | Isolate Bl: 36 EU | | |
| 64. | Resistance to Lettuce root aphid | absent | absent |
| | (Pb) | | |
| 65. | Resistance to Lettuce Necrotic | absent | absent |
| | Stunt Virus LNSV | | |
| 66. | Resistance to Lettuce Big Vein | absent | absent |
| | Virus LBVV | | |
| 67. | Head: diameter of the cut of the | big | big |
| | trunk | | |
| 68. | Head: height of the bottom ribs | medium | medium |

### Lettuce Variety ‘1434369’

Provided herein is lettuce variety ‘1434369,’ a crisphead lettuce variety suitable for production in the spring and autumn harvesting seasons in, for example, Central America (e.g., Guatemala). Lettuce variety ‘1434369’ is the result of a cross between a mother crisphead line that was susceptible to Bremia lactucae and a Bremia lactucae-resistant crisphead line, and was selected following backcrosses and selfing. These plants were chosen for their dark green leaf color and resistance to Bremia lactucae.

Lettuce variety ‘1434369’ has uniformity and stability for these traits, within the limits of environmental influence for the traits. It has been self-pollinated through a sufficient number of generations with careful attention to uniformity of plant type. The variety has been propagated with continuous attention for uniformity.

### Breeding History

Crosses were made by spraying or misting water over the flowers, thereby inactivating pollen present on these flowers. This treatment enables the cross pollination of Lactuca plants.

F1 plants can be self-pollinated to produce the F2 generation, etc.

A Bremia lactucae-susceptible crisphead line was used as the mother variety, and was crossed with a Bremia lactucae-resistant line. The Bremia lactucae-resistant line is described in U.S. Pat. No. 10,575,479 and U.S. Patent Application Publication No. 2020/0029523, both of which are incorporated by reference herein in their entirety.

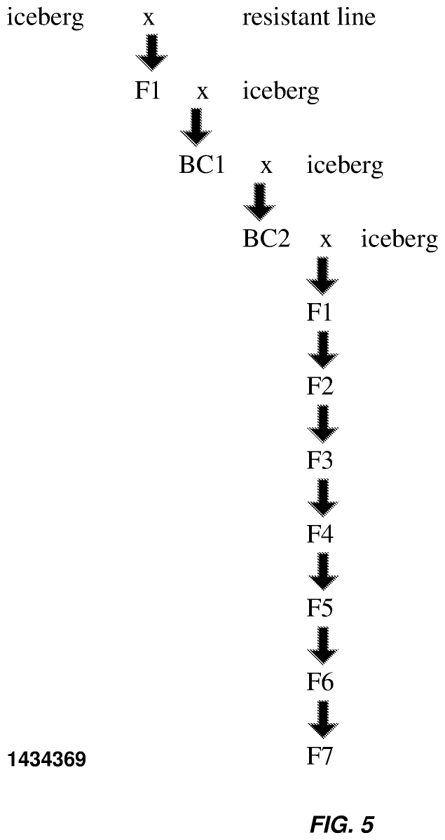

From these crosses, the F1 plants with the greatest resistance to Bremia lactucae were identified and a single plant was selected and backcrossed with the mother line two times. Following the backcrosses, the last, best-performing generation was selfed for seven generations. The best-performing line was then selected (FIG. 5).

Seeds of all plants from this most-uniform line were collectively harvested and designated crisphead variety ‘1434369’. A deposit of seeds of ‘1434369’ as disclosed above and recited in the appended claims has been made with the National Collections of Industrial, Food and Marine Bacteria (NCIMB), Ferguson Building, Craibstone Estate, Bucksburn, Aberdeen AB21 9YA, United Kingdom. The date of the deposit was Feb. 7, 2020. All restrictions will be irrevocably removed upon granting of a patent, and the deposit is intended to meet all of the requirements of 37 C.F.R. § 1.801-1.809. The NCIMB Accession Number is 43573. The deposit will be maintained in the depository for a period of thirty years, or five years after the last request, or for the enforceable life of the patent, whichever is longer, and will be replaced as necessary during that period.

### Variety Description

Lettuce variety ‘1434369’ is a seed-propagated crisphead lettuce which is open pollinated. Variety ‘1434369’ is resistant to at least Bremia lactucae isolates Bl:16-36 and Nasonovia ribisnigri:0. Lettuce variety ‘1434369’ has black seeds, exhibits dark green leaves and medium to late bolting under long day conditions, and is comparable to the variety ‘Cartagenas’. With regard to this comparison, variety ‘1434369’ exhibits medium slow bolting (compared to slow bolting for ‘Cartagenas’), is larger than ‘Cartagenas,’ and has medium flat ribs (compared to the flat ribs of ‘Cartagenas’).

Characteristics of ‘1434369’ in concordance with the applicable UPOV Questionnaire, as compared to the ‘Cartagenas’ variety, are provided below in Table 5.

TABLE 5

| | Characteristic | ‘1434369’ | | ‘Cartagenas’ | |
|---|---|---|---|---|---|
| 1. | Seed: color | black | | black | |
| 2. | Plant: diameter (at market stage) | 45-50 | cm | 35-40 | cm |
| 3. | Plant: degree of overlapping of | strong | | strong | |
| | upper part of leaves | | | | |
| 4. | Leaf: number of leaves | not applicable | | not applicable | |
| 5. | Leaf: attitude | semi-erect | | semi-erect | |
| 6. | Leaf: number of divisions | absent or | | absent or | |
| | | very few | | very few | |
| 7. | Leaf: shape | medium oblate | | broad obtrullate | |
| 8. | Leaf: shape of apex | obcordate | | obcordate | |
| 9. | Leaf: longitudinal section | convex | | convex | |
| 10. | Leaf: width or lobes [only | not applicable | | not applicable | |
| | oakleaf type] | | | | |
| 11. | Leaf: anthocyanin coloration | absent or | | absent or | |
| | | very weak | | very weak | |
| 12. | Leaf: hue of anthocyanin | not applicable | | not applicable | |
| | coloration | | | | |
| 13. | Leaf: area covered by anthocyanin | very small | | very small | |
| | coloration | | | | |
| 14. | Leaf: color | green | | green | |
| 15. | Leaf: intensity of green color | very dark | | very dark | |
| 16. | Leaf: glossiness of upper side | medium | | medium | |
| 17. | Leaf: thickness | thick | | thick | |
| 18. | Leaf: blistering | absent or | | absent or | |
| | | very weak | | very weak | |
| 19. | Leaf: size of blisters | large | | large | |
| 20. | Leaf: undulation of margin | weak | | weak | |
| 21. | Leaf: type of incisions of margin | regularly | | irregularly | |
| | | dendate | | dendate | |
| 22. | Leaf: depth of incisions of margin | absent or | | absent or | |
| | | very shallow | | very shallow | |
| 23. | Leaf: depth of secondary incisions | not applicable | | not applicable | |
| | of margin | | | | |
| 24. | Leaf: density of incisions of | very sparse | | very sparse | |
| | margin | | | | |
| 25. | Leaf: venation | flabellate | | flabellate | |
| 26. | Head: size | large | | large | |
| 27. | Head: shape in longitudinal | circular | | circular | |
| | section | | | | |
| 28. | Head: density | medium | | medium | |
| 29. | Stem: length | not applicable | | not applicable | |
| 30. | Stem: width | not applicable | | not applicable | |
| 31. | Stem: shape in longitudinal section | not applicable | | not applicable | |
| 32. | Stem: color | not applicable | | not applicable | |
| 33. | Stem: color of flesh | not applicable | | not applicable | |
| 34. | Time of harvest maturity | medium | | medium | |
| 35. | Time of beginning of bolting | medium | | late | |
| 36. | Axillary sprouting | absent or weak | | absent or weak | |
| 37. | Bolting stem: fasciation | absent or weak | | absent or weak | |
| 38. | Resistance to Bremia lactucae (Bl) | Present | | Present | |
| | Isolate Bl: 16EU | | | | |
| 39. | Resistance to Bremia lactucae (Bl) | Present | | Absent | |
| | Isolate Bl: 17EU | | | | |
| 40. | Resistance to Bremia lactucae (Bl) | Present | | Absent | |
| | Isolate Bl: 20EU | | | | |
| 41. | Resistance to Bremia lactucae (Bl) | Present | | Present | |
| | Isolate Bl: 21EU | | | | |
| 42. | Resistance to Bremia lactucae (Bl) | Present | | Absent | |
| | Isolate Bl: 22EU | | | | |
| 43. | Resistance to Bremia lactucae (Bl) | Present | | Present | |
| | Isolate Bl: 23EU | | | | |
| 44. | Resistance to Bremia lactucae (Bl) | Present | | Absent | |
| | Isolate Bl: 24EU | | | | |
| 45. | Resistance to Bremia lactucae (Bl) | Present | | Absent | |
| | Isolate Bl: 25EU | | | | |
| 46. | Resistance to Bremia lactucae (Bl) | Present | | Absent | |
| | Isolate Bl: 26EU | | | | |
| 47. | Resistance to Bremia lactucae (Bl) | Present | | Absent | |
| | Isolate Bl: 27EU | | | | |
| 48. | Resistance to Bremia lactucae (Bl) | Present | | Absent | |
| | Isolate Bl: 29EU | | | | |
| 49. | Resistance to Bremia lactucae (Bl) | Present | | Absent | |
| | Isolate Bl: 30EU | | | | |
| 50. | Resistance to Bremia lactucae (Bl) | Present | | Absent | |
| | Isolate Bl: 31EU | | | | |
| 51. | Resistance to Bremia lactucae (Bl) | Present | | Absent | |
| | Isolate Bl: 33EU | | | | |
| 52. | Resistance to Bremia lactucae (Bl) | Present | | Absent | |
| | Isolate Bl: 35EU | | | | |
| 53. | Resistance to Lettuce mosaic virus | absent | | absent | |
| | (LMV) Pathotype II | | | | |
| 54. | Resistance to Nasonovia ribisnigri | Present | | Present | |
| | (Nr) | | | | |
| | Biotype Nr: 0 | | | | |
| 55. | Resistance to Fusarium | susceptible | | susceptible | |
| | oxysporum f. sp. lactucae (Fol) | | | | |
| | Race 1 | | | | |
| 56. | Head: size class | 9-10/box | | 9-10/box | |
| 57. | Head: Shape | balround | | balround | |
| 58. | Head: leaf wrap over the top of the | medium | | medium | |
| | head | | | | |
| 59. | Head: height head (mature stage; | 12.0 | cm | 11.0 | cm |
| | from cut till top) | | | | |
| 60. | Head: weight (mature stage; | 742 | gr | 668 | gr |
| | average of 4-5) | | | | |

| | | | |
|---|---|---|---|
| 61. | Resistance to Bremia lactucae (Bl) | present | present |
| | Isolate Bl: 32 EU | | |
| 62 | Resistance to Bremia lactucae (Bl) | present | absent |
| | Isolate Bl: 34 EU | | |
| 63. | Resistance to Bremia lactucae (Bl) | present | absent |
| | Isolate Bl: 36 EU | | |
| 64. | Resistance to Lettuce root aphid | absent | absent |
| | (Pb) | | |
| 65. | Resistance to Lettuce Necrotic | absent | absent |
| | Stunt Virus LNSV | | |
| 66. | Resistance to Lettuce Big Vein | absent | absent |
| | Virus LBVV | | |
| 67. | Head: diameter of the cut of the | big | big |
| | trunk | | |
| 68. | Head: height of the bottom ribs | medium | low |

### Lettuce Variety ‘1434370’

Provided herein is lettuce variety ‘1434370,’ a crisphead lettuce variety suitable for production in the spring and autumn harvesting seasons in, for example, Northern Europe. Lettuce variety ‘1434370’ is the result of a cross between a mother crisphead line that was susceptible to Bremia lactucae and a Bremia lactucae-resistant crisphead line, and was selected following backcrossing and selfing. These plants were chosen for their dark to very dark green leaf color and resistance to Bremia lactucae.

Lettuce variety ‘1434370’ has uniformity and stability for these traits, within the limits of environmental influence for the traits. It has been self-pollinated through a sufficient number of generations with careful attention to uniformity of plant type. The variety has been propagated with continuous attention for uniformity.

### Breeding History

Crosses were made by spraying or misting water over the flowers, thereby inactivating pollen present on these flowers. This treatment enables the cross pollination of Lactuca plants.

F1 plants can be self-pollinated to produce the F2 generation, etc.

A Bremia lactucae-susceptible crisphead line was used as the mother variety, and was crossed with a Bremia lactucae-resistant line. The Bremia lactucae-resistant line is described in U.S. Pat. No. 10,575,479 and U.S. Patent Application Publication No. 2020/0029523, both of which are incorporated by reference herein in their entirety.

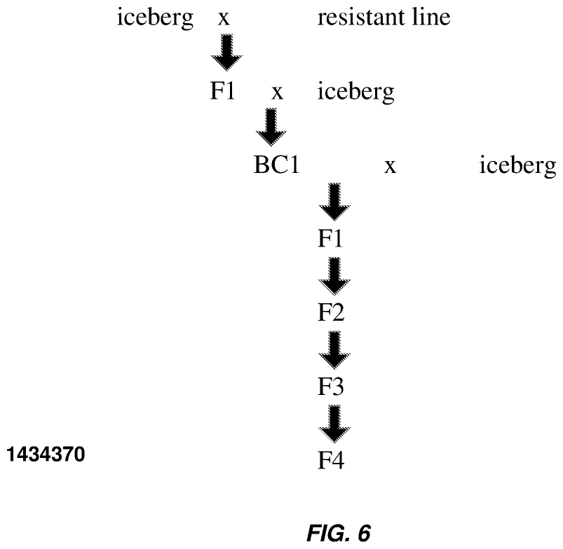

From these crosses, the F1 plants with the greatest resistance to Bremia lactucae were identified and a single plant was selected and backcrossed with the mother line. Following the backcross, the best-performing line was selfed for four generations. The best-performing line was then selected (FIG. 6).

Seeds of all plants from this most-uniform line were collectively harvested and designated crisphead variety ‘1434370’. A deposit of seeds of ‘1434370’ as disclosed above and recited in the appended claims has been made with the National Collections of Industrial, Food and Marine Bacteria. (NCIMB), Ferguson Building, Craibstone Estate, Bucksburn, Aberdeen AB21 9YA, United Kingdom. The date of the deposit was Feb. 7, 2020. All restrictions will be irrevocably removed upon granting of a patent, and the deposit is intended to meet all of the requirements of 37 C.F.R. § 1.801-1.809. The NCIMB Accession Number is 43571. The deposit will be maintained in the depository for a period of thirty years, or five years after the last request, or for the enforceable life of the patent, whichever is longer, and will be replaced as necessary during that period.

### Variety Description

Lettuce variety ‘1434370’ is a seed-propagated crisphead lettuce which is open pollinated. Variety ‘1434370’ is resistant to at least Bremia lactucae isolates Bl:16-36 and Nasonovia ribisnigri:0. Lettuce variety ‘1434370’ has black seeds, exhibits dark green leaves and late bolting under long day conditions, and is comparable to the variety ‘Rumours’. With regard to this comparison, variety ‘1434370’ exhibits a flatround head (compared to a round head for ‘Rumours’) and is larger than ‘Rumours’.

Characteristics of ‘1434370’ in concordance with the applicable UPOV Questionnaire, as compared to the ‘Rumours’ variety, are provided below in Table 6.

TABLE 6

| | Characteristic | ‘1434370’ | | ‘Rumours’ | |
|---|---|---|---|---|---|
| 1. | Seed: color | black | | black | |
| 2. | Plant: diameter (at market stage) | 40-45 | cm | 35-40 | cm |
| 3. | Plant: degree of overlapping of | strong | | strong | |
| | upper part of leaves | | | | |
| 4. | Leaf: number of leaves | not applicable | | not applicable | |
| 5. | Leaf: attitude | semi-erect | | semi-erect | |
| 6. | Leaf: number of divisions | absent or | | absent or | |
| | | very few | | very few | |
| 7. | Leaf: shape | narrow | | broad | |
| | | oblate | | obtrullate | |
| 8. | Leaf: shape of apex | obcordate | | obcordate | |
| 9. | Leaf: longitudinal section | convex | | convex | |
| 10. | Leaf: width or lobes [only | not applicable | | not applicable | |
| | oakleaf type] | | | | |
| 11. | Leaf: anthocyanin coloration | absent or | | absent or | |
| | | very weak | | very weak | |
| 12. | Leaf: hue of anthocyanin | not applicable | | not applicable | |
| | coloration | | | | |
| 13. | Leaf: area covered by anthocyanin | very small | | very small | |
| | coloration | | | | |
| 14. | Leaf: color | green | | green | |
| 15. | Leaf: intensity of green color | dark | | very dark | |
| 16. | Leaf: glossiness of upper side | strong | | strong | |
| 17. | Leaf: thickness | thick | | thick | |
| 18. | Leaf: blistering | absent or | | absent or | |
| | | very weak | | very weak | |
| 19. | Leaf: size of blisters | large | | large | |
| 20. | Leaf: undulation of margin | Absent or | | Absent or | |
| | | very weak | | very weak | |
| 21. | Leaf: type of incisions of | irregularly | | irregularly | |
| | margin | dendate | | dendate | |
| 22. | Leaf: depth of incisions of | medium | | medium | |
| | margin | | | | |
| 23. | Leaf: depth of secondary | not applicable | | not applicable | |
| | incisions of margin | | | | |
| 24. | Leaf: density of incisions of | sparse | | sparse | |
| | margin | | | | |
| 25. | Leaf: venation | flabellate | | flabellate | |
| 26. | Head: size | large | | medium | |
| 27. | Head: shape in longitudinal section | narrow oblate | | circular | |
| 28. | Head: density | medium | | dense | |
| 29. | Stem: length | not applicable | | not applicable | |
| 30. | Stem: width | not applicable | | not applicable | |
| 31. | Stem: shape in longitudinal section | not applicable | | not applicable | |
| 32. | Stem: color | not applicable | | not applicable | |
| 33. | Stem: color of flesh | not applicable | | not applicable | |
| 34. | Time of harvest maturity | medium | | early | |
| 35. | Time of beginning of bolting | medium | | late | |
| 36. | Axillary sprouting | absent or weak | | medium | |
| 37. | Bolting stem: fasciation | absent or | | absent or | |
| | | very weak | | very weak | |
| 38. | Resistance to Bremia lactucae (Bl) | Present | | Present | |
| | Isolate Bl: 16EU | | | | |
| 39. | Resistance to Bremia lactucae (Bl) | Present | | Present | |
| | Isolate Bl: 17EU | | | | |
| 40. | Resistance to Bremia lactucae (Bl) | Present | | Present | |
| | Isolate Bl: 20EU | | | | |
| 41. | Resistance to Bremia lactucae (Bl) | Present | | Present | |
| | Isolate Bl: 21EU | | | | |
| 42. | Resistance to Bremia lactucae (Bl) | Present | | Present | |
| | Isolate Bl: 22EU | | | | |
| 43. | Resistance to Bremia lactucae (Bl) | Present | | Present | |
| | Isolate Bl: 23EU | | | | |
| 44. | Resistance to Bremia lactucae (Bl) | Present | | Present | |
| | Isolate Bl: 24EU | | | | |
| 45. | Resistance to Bremia lactucae (Bl) | Present | | Present | |
| | Isolate Bl: 25EU | | | | |
| 46. | Resistance to Bremia lactucae (Bl) | Present | | Present | |
| | Isolate Bl: 26EU | | | | |
| 47. | Resistance to Bremia lactucae (Bl) | Present | | Present | |
| | Isolate Bl: 27EU | | | | |
| 48. | Resistance to Bremia lactucae (Bl) | Present | | Present | |
| | Isolate Bl: 29EU | | | | |
| 49. | Resistance to Bremia lactucae (Bl) | Present | | Present | |
| | Isolate Bl: 30EU | | | | |
| 50. | Resistance to Bremia lactucae (Bl) | Present | | Present | |
| | Isolate Bl: 31EU | | | | |
| 51. | Resistance to Bremia lactucae (Bl) | Present | | Present | |
| | Isolate Bl: 33EU | | | | |
| 52. | Resistance to Bremia lactucae (Bl) | Present | | Present | |
| | Isolate Bl: 35EU | | | | |
| 53. | Resistance to Lettuce mosaic virus | absent | | Present | |
| | (LMV) Pathotype II | | | | |
| 54. | Resistance to Nasonovia ribisnigri | Present | | Present | |
| | (Nr) | | | | |
| | Biotype Nr: 0 | | | | |
| 55. | Resistance to Fusarium oxysporum | susceptible | | Moderately | |
| | f. sp. lactucae (Fol) Race 1 | | | resistant | |
| 56. | Head: size class | 9-10/box | | 10-11/box | |
| 57. | Head: Shape | Flatround | | Balround | |
| 58. | Head: leaf wrap over the top of the | medium | | medium | |
| | head | | | | |
| 59. | Head: heighy head (mature stage; | 10.5 | cm | 11.0 | cm |
| | from cut till top) | | | | |
| 60. | Head: weight (mature stage; | 403 | gr | 543 | gr |
| | average of 4-5) | | | | |

| | | | |
|---|---|---|---|
| 61. | Resistance to Bremia lactucae (Bl) | Present | Present |
| | Isolate Bl: 32 EU | | |
| 62 | Resistance to Bremia lactucae (Bl) | Present | Present |
| | Isolate Bl: 34 EU | | |
| 63. | Resistance to Bremia lactucae (Bl) | Present | Present |
| | Isolate Bl: 36 EU | | |
| 64. | Resistance to Lettuce root aphid | Absent | Absent |
| | (Pb) | | |
| 65. | Resistance to Lettuce Necrotic | Absent | Absent |
| | Stunt Virus LNSV | | |
| 66. | Resistance to Lettuce Big Vein | Absent | Absent |
| | Virus LBVV | | |
| 67. | Head: diameter of the cut of the | Medium | big |
| | trunk | | |
| 68. | Head: height of the bottom ribs | low | Medium |

### Lettuce Variety ‘1434371’

Provided herein is lettuce variety ‘1434371,’ a crisphead lettuce variety suitable for production in the spring and winter harvesting seasons in, for example, Spain. Lettuce variety ‘1434371’ is the result of a cross between a mother crisphead line that was susceptible to Bremia lactucae and a Bremia lactucae-resistant crisphead line, and was selected following backcrosses and selfing. These plants were chosen for their dark to very dark green leaf color and resistance to Bremia lactucae.

Lettuce variety ‘1434371’ has uniformity and stability for these traits, within the limits of environmental influence for the traits. It has been self-pollinated through a sufficient number of generations with careful attention to uniformity of plant type. The variety has been propagated with continuous attention for uniformity.

### Breeding History

Crosses were made by spraying or misting water over the flowers, thereby inactivating pollen present on these flowers. This treatment enables the cross pollination of Lactuca plants.

F1 plants can be self-pollinated to produce the F2 generation, etc.

A Bremia lactucae-susceptible crisphead line was used as the mother variety, and was crossed with a Bremia lactucae-resistant line. The Bremia lactucae-resistant line is described in U.S. Pat. No. 10,575,479 and U.S. Patent Application Publication No. 2020/0029523, both of which are incorporated by reference herein in their entirety.

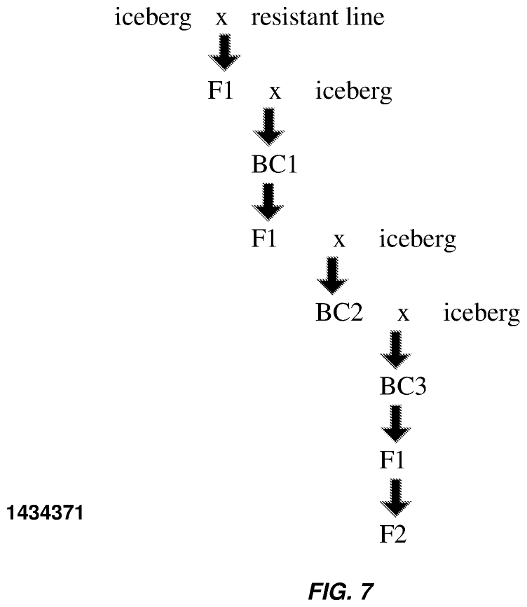

From these crosses, the F1 plants with the greatest resistance to Bremia lactucae were identified and a single plant was selected and backcrossed with the mother line. Following the backcross, the best-performing line was selected in the field, and backcrossed twice with the mother line and selfed for two generations. The best-performing line was then selected (FIG. 7).

Seeds of all plants from this most-uniform line were collectively harvested and designated crisphead variety ‘1434371’. A deposit of seeds of ‘1434371’ as disclosed above and recited in the appended claims has been made with the National Collections of industrial, Food and Marine Bacteria. (NCIMB), Ferguson Building, Craibstone Estate, Bucksburn, Aberdeen AB21 9YA, United Kingdom. The date of the deposit was Feb. 7, 2020. All restrictions will be irrevocably removed upon granting of a patent, and the deposit is intended to meet all of the requirements of 37 C.F.R. § 1.801-1.809. The NCIMB Accession Number is 43570. The deposit will be maintained in the depository for a period of thirty years, or five years after the last request, or for the enforceable life of the patent, whichever is longer, and will be replaced as necessary during that period.

### Variety Description

Lettuce variety ‘1434371’ is a seed-propagated crisphead lettuce which is open pollinated. Variety ‘1434371’ is resistant to at least Bremia lactucae isolates Bl:16-36 and Nasonovia ribisnigri:0. Lettuce variety ‘1434371’ has black seeds, exhibits dark to very dark green leaves and late bolting under long day conditions, and is comparable to the variety ‘Diurnas’. With regard to this comparison, variety ‘1434371’ exhibits a round head (compared to a flat round head for ‘Diurnas’), medium flat ribs (compared to flat ribs for ‘Diurnas’), darker leaves, and has a thick leaf (compared to a medium thick leaf for ‘Diurnas’).

Characteristics of ‘1434371’ in concordance with the applicable UPOV Questionnaire, as compared to the ‘Diurnas’ variety, are provided below in Table 7.

TABLE 7

| | Characteristic | ‘1434371’ | | ‘Diurnas’ | |
|---|---|---|---|---|---|
| 1. | Seed: color | black | | black | |
| 2. | Plant: diameter (at market stage) | 35-38 | cm | 38-40 | cm |
| 3. | Plant: degree of overlapping of | strong | | strong | |
| | upper part of leaves | | | | |
| 4. | Leaf: number of leaves | not applicable | | not applicable | |
| 5. | Leaf: attitude | semi-erect | | semi-erect | |
| 6. | Leaf: number of divisions | absent or | | absent or | |
| | | very few | | very few | |
| 7. | Leaf: shape | broad | | broad | |
| | | obtrullate | | obtrullate | |
| 8. | Leaf: shape of apex | obcordate | | obcordate | |
| 9. | Leaf: longitudinal section | convex | | convex | |
| 10. | Leaf: width or lobes [only | not applicable | | not applicable | |
| | oakleaf type] | | | | |
| 11. | Leaf: anthocyanin coloration | absent or | | absent or | |
| | | very weak | | very weak | |
| 12. | Leaf: hue of anthocyanin | not applicable | | not applicable | |
| | coloration | | | | |
| 13. | Leaf: area covered by anthocyanin | very small | | very small | |
| | coloration | | | | |
| 14. | Leaf: color | green | | green | |
| 15. | Leaf: intensity of green color | very dark | | dark | |
| 16. | Leaf: glossiness of upper side | medium | | medium | |
| 17. | Leaf: thickness | thick | | thick | |
| 18. | Leaf: blistering | absent or | | absent or | |
| | | very weak | | very weak | |
| 19. | Leaf: size of blisters | large | | large | |
| 20. | Leaf: undulation of margin | weak | | weak | |
| 21. | Leaf: type of incisions of | irregularly | | irregularly | |
| | margin | dendate | | dendate | |
| 22. | Leaf: depth of incisions of | shallow | | absent or | |
| | margin | | | very shallow | |
| 23. | Leaf: depth of secondary incisions | not applicable | | not applicable | |
| | of margin | | | | |
| 24. | Leaf: density of incisions of | very sparse | | very sparse | |
| | margin | | | | |
| 25. | Leaf: venation | flabellate | | flabellate | |
| 26. | Head: size | large | | large | |
| 27. | Head: shape in longitudinal | narrow oblate | | narrow oblate | |
| | section | | | | |
| 28. | Head: density | dense | | dense | |
| 29. | Stem: length | not applicable | | not applicable | |
| 30. | Stem: width | not applicable | | not applicable | |
| 31. | Stem: shape in longitudinal section | not applicable | | not applicable | |
| 32. | Stem: color | not applicable | | not applicable | |
| 33. | Stem: color of flesh | not applicable | | not applicable | |
| 34. | Time of harvest maturity | medium | | medium | |
| 35. | Time of beginning of bolting | medium | | medium | |
| 36. | Axillary sprouting | weak | | absent or weak | |
| 37. | Bolting stem: fasciation | absent or | | absent or | |
| | | very weak | | very weak | |
| 38. | Resistance to Bremia lactucae (Bl) | Present | | Present | |
| | Isolate Bl: 16EU | | | | |
| 39. | Resistance to Bremia lactucae (Bl) | Present | | Present | |
| | Isolate Bl: 17EU | | | | |
| 40. | Resistance to Bremia lactucae (Bl) | Present | | Present | |
| | Isolate Bl: 20EU | | | | |
| 41. | Resistance to Bremia lactucae (Bl) | Present | | Present | |
| | Isolate Bl: 21EU | | | | |
| 42. | Resistance to Bremia lactucae (Bl) | Present | | Present | |
| | Isolate Bl: 22EU | | | | |
| 43. | Resistance to Bremia lactucae (Bl) | Present | | Present | |
| | Isolate Bl: 23EU | | | | |
| 44. | Resistance to Bremia lactucae (Bl) | Present | | Present | |
| | Isolate Bl: 24EU | | | | |
| 45. | Resistance to Bremia lactucae (Bl) | Present | | Present | |
| | Isolate Bl: 25EU | | | | |
| 46. | Resistance to Bremia lactucae (Bl) | Present | | Present | |
| | Isolate Bl: 26EU | | | | |
| 47. | Resistance to Bremia lactucae (Bl) | Present | | Present | |
| | Isolate Bl: 27EU | | | | |
| 48. | Resistance to Bremia lactucae (Bl) | Present | | Present | |
| | Isolate Bl: 29EU | | | | |
| 49. | Resistance to Bremia lactucae (Bl) | Present | | Present | |
| | Isolate Bl: 30EU | | | | |
| 50. | Resistance to Bremia lactucae (Bl) | Present | | Present | |
| | Isolate Bl: 31EU | | | | |
| 51. | Resistance to Bremia lactucae (Bl) | Present | | Present | |
| | Isolate Bl: 33EU | | | | |
| 52. | Resistance to Bremia lactucae (Bl) | Present | | Present | |
| | Isolate Bl: 35EU | | | | |
| 53. | Resistance to Lettuce mosaic virus | Absent | | Absent | |
| | (LMV) Pathotype II | | | | |
| 54. | Resistance to Nasonovia ribisnigri | Present | | Present | |
| | (Nr) | | | | |
| | Biotype Nr: 0 | | | | |
| 55. | Resistance to Fusarium | susceptible | | susceptible | |
| | oxysporum f. sp. lactucae (Fol) | | | | |
| | Race 1 | | | | |
| 56. | Head: size class | 9-10/box | | 8-10/box | |
| 57. | Head: Shape | Balround | | Little | |
| | | | | flatround | |
| 58. | Head: leaf wrap over the top of the | medium | | medium | |
| | head | | | | |
| 59. | Head: height head (mature stage; | 12.5 | cm | 10.5 | cm |
| | from cut till top) | | | | |
| 60. | Head: weight (mature stage; | 544 | gr | 404 | gr |
| | average of 4-5) | | | | |

| | | | |
|---|---|---|---|
| 61. | Resistance to Bremia lactucae (Bl) | Present | Present |
| | Isolate Bl: 32 EU | | |
| 62 | Resistance to Bremia lactucae (Bl) | Present | Present |
| | Isolate Bl: 34 EU | | |
| 63. | Resistance to Bremia lactucae (Bl) | Present | Present |
| | Isolate Bl: 36 EU | | |
| 64. | Resistance to Lettuce root aphid | Absent | Absent |
| | (Pb) | | |
| 65. | Resistance to Lettuce Necrotic | Absent | Absent |
| | Stunt Virus LNSV | | |
| 66. | Resistance to Lettuce Big Vein | Absent | Absent |
| | Virus LBVV | | |
| 67. | Head: diameter of the cut of the | big | medium |
| | trunk | | |
| 68. | Head: height of the bottom ribs | medium | medium |

### Lettuce Variety ‘1434377’

Provided herein is lettuce variety ‘1434377,’ a crisphead lettuce variety suitable for production in the spring and autumn harvesting seasons in, for example, Northern Europe. Lettuce variety ‘1434377’ is the result of a cross between a mother crisphead line that was susceptible to Bremia lactucae and a Bremia lactucae-resistant crisphead line, and was selected following a backcross and selfing. These plants were chosen for their dark to very dark green leaf color and resistance to Bremia lactucae.

Lettuce variety ‘1434377’ has uniformity and stability for these traits, within the limits of environmental influence for the traits. It has been self-pollinated through a sufficient number of generations with careful attention to uniformity of plant type. The variety has been propagated with continuous attention for uniformity.

### Breeding History

Crosses were made by spraying or misting water over the flowers, thereby inactivating pollen present on these flowers. This treatment enables the cross pollination of Lactuca plants.

F1 plants can be self-pollinated to produce the F2 generation, etc.

A Bremia lactucae-susceptible crisphead line was used as the mother variety, and was crossed with a Bremia lactucae-resistant line. The Bremia lactucae-resistant line is described in U.S. Pat. No. 10,575,479 and U.S. Patent Application Publication No. 2020/0029523, both of which are incorporated by reference herein in their entirety.

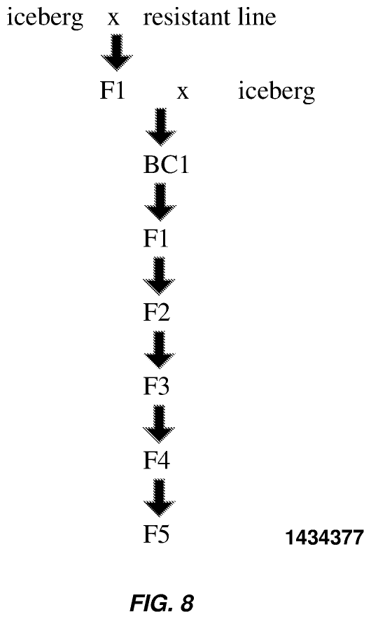

From these crosses, the F1 plants with the greatest resistance to Bremia lactucae were identified and a single plant was selected and backcrossed with the mother line. Following the backcross, the best-performing line was selected in the field, and backcrossed twice with the mother line and selfed for two generations. The best-performing line was then selected (FIG. 8).

Seeds of all plants from this most-uniform line were collectively harvested and designated crisphead variety ‘1434377’. A deposit of seeds of ‘1434377’ as disclosed above and recited in the appended claims has been made with the National Collections of Industrial, Food and Marine Bacteria (NCIMB), Ferguson Building, Craibstone Estate, Bucksburn, Aberdeen AB21 9YA, United Kingdom. The date of the deposit was Feb. 7, 2020. All restrictions will be irrevocably removed upon granting of a patent, and the deposit is intended to meet all of the requirements of 37 C.F.R. § 1.8014.809. The NCIMB Accession Number is 43569. The deposit will be maintained in the depository for a period of thirty years, or five years after the last request, or for the enforceable life of the patent, whichever is longer, and will be replaced as necessary during that period.

### Variety Description

Lettuce variety ‘1434377’ is a seed-propagated crisphead lettuce which is open pollinated. Variety ‘1434377’ is resistant to at least Bremia lactucae isolates Bl:16-36, Nasonovia ribisnigri:0, and LMV Strain LS-1. Lettuce variety ‘1434377’ has black seeds, exhibits dark to very dark green leaves and late bolting under long day conditions, and is comparable to the variety ‘Rumours’. With regard to this comparison, variety ‘1434377’ exhibits a larger head, a slightly flat round head shape (compared to round for ‘Rumours’), and dark green leaves (compared to very dark green for ‘Rumours’).

Characteristics of ‘1434377’ in concordance with the applicable UPOV Questionnaire, as compared to the ‘Rumours’ variety, are provided below in Table 8.

TABLE 8

| | Characteristic | ‘1434377’ | | ‘Rumours’ | |
|---|---|---|---|---|---|
| 1. | Seed: color | black | | black | |
| 2. | Plant: diameter (at market stage) | 40-45 | cm | 35-40 | cm |
| 3. | Plant: degree of overlapping of | strong | | strong | |
| | upper part of leaves | | | | |
| 4. | Leaf: number of leaves | not applicable | | not applicable | |
| 5. | Leaf: attitude | semi-erect | | semi-erect | |
| 6. | Leaf: number of divisions | absent or | | absent or | |
| | | very few | | very few | |
| 7. | Leaf: shape | broad obtrullate | | broad obtrullate | |
| 8. | Leaf: shape of apex | obcordate | | obcordate | |
| 9. | Leaf: longitudinal section | convex | | convex | |
| 10. | Leaf: width or lobes [only | not applicable | | not applicable | |
| | oakleaf type] | | | | |
| 11. | Leaf: anthocyanin coloration | absent or | | absent or | |
| | | very weak | | very weak | |
| 12. | Leaf: hue of anthocyanin | not applicable | | not applicable | |
| | coloration | | | | |
| 13. | Leaf: area covered by anthocyanin | very small | | very small | |
| | coloration | | | | |
| 14. | Leaf: color | green | | green | |
| 15. | Leaf: intensity of green color | dark | | very dark | |
| 16. | Leaf: glossiness of upper side | medium | | strong | |
| 17. | Leaf: thickness | thick | | thick | |
| 18. | Leaf: blistering | absent or | | absent or | |
| | | very weak | | very weak | |
| 19. | Leaf: size of blisters | Medium | | large | |
| 20. | Leaf: undulation of margin | Absent or | | Absent or | |
| | | very weak | | very weak | |
| 21. | Leaf: type of incisions of margin | irregularly | | irregularly | |
| | | dendate | | dendate | |
| 22. | Leaf: depth of incisions of margin | medium | | medium | |
| 23. | Leaf: depth of secondary incisions | not | | not | |
| | of margin | applicable | | applicable | |
| 24. | Leaf: density of incisions of | sparse | | sparse | |
| | margin | | | | |
| 25. | Leaf: venation | flabellate | | flabellate | |
| 26. | Head: size | large | | medium | |
| 27. | Head: shape in longitudinal | narrow oblate | | circular | |
| | section | | | | |
| 28. | Head: density | medium | | dense | |
| 29. | Stem: length | not applicable | | not applicable | |
| 30. | Stem: width | not applicable | | not applicable | |
| 31. | Stem: shape in longitudinal section | not applicable | | not applicable | |
| 32. | Stem: color | not applicable | | not applicable | |
| 33. | Stem: color of flesh | not applicable | | not applicable | |
| 34. | Time of harvest maturity | medium | | early | |
| 35. | Time of beginning of bolting | late | | late | |
| 36. | Axillary sprouting | absent or weak | | medium | |
| 37. | Bolting stem: fasciation | absent or | | absent or | |
| | | very weak | | very weak | |
| 38. | Resistance to Bremia lactucae (Bl) | Present | | Present | |
| | Isolate Bl: 16EU | | | | |
| 39. | Resistance to Bremia lactucae (Bl) | Present | | Present | |
| | Isolate Bl: 17EU | | | | |
| 40. | Resistance to Bremia lactucae (Bl) | Present | | Present | |
| | Isolate Bl: 20EU | | | | |
| 41. | Resistance to Bremia lactucae (Bl) | Present | | Present | |
| | Isolate Bl: 21EU | | | | |
| 42. | Resistance to Bremia lactucae (Bl) | Present | | Present | |
| | Isolate Bl: 22EU | | | | |
| 43. | Resistance to Bremia lactucae (Bl) | Present | | Present | |
| | Isolate Bl: 23EU | | | | |
| 44. | Resistance to Bremia lactucae (Bl) | Present | | Present | |
| | Isolate Bl: 24EU | | | | |
| 45. | Resistance to Bremia lactucae (Bl) | Present | | Present | |
| | Isolate Bl: 25EU | | | | |
| 46. | Resistance to Bremia lactucae (Bl) | Present | | Present | |
| | Isolate Bl: 26EU | | | | |
| 47. | Resistance to Bremia lactucae (Bl) | Present | | Present | |
| | Isolate Bl: 27EU | | | | |
| 48. | Resistance to Bremia lactucae (Bl) | Present | | Present | |
| | Isolate Bl: 29EU | | | | |
| 49. | Resistance to Bremia lactucae (Bl) | Present | | Present | |
| | Isolate Bl: 30EU | | | | |
| 50. | Resistance to Bremia lactucae (Bl) | Present | | Present | |
| | Isolate Bl: 31EU | | | | |
| 51. | Resistance to Bremia lactucae (Bl) | Present | | Present | |
| | Isolate Bl: 33EU | | | | |
| 52. | Resistance to Bremia lactucae (Bl) | Present | | Present | |
| | Isolate Bl: 35EU | | | | |
| 53. | Resistance to Lettuce mosaic virus | Present | | Present | |
| | (LMV) Pathotype II | | | | |
| 54. | Resistance to Nasonovia ribisnigri | Present | | Present | |
| | (Nr) | | | | |
| | Biotype Nr: 0 | | | | |
| 55. | Resistance to Fusarium | susceptible | | Moderately | |
| | oxysporum f. sp. lactucae (Fol) | | | resistant | |
| | Race 1 | | | | |
| 56. | Head: size class | 10/box | | 10-11/box | |
| 57. | Head: Shape | Flatround | | Balround | |
| 58. | Head: leaf wrap over the top of the | medium | | medium | |
| | head | | | | |
| 59. | Head: height head (mature stage; | 11.0 | cm | 11.0 | cm |
| | from cut till top) | | | | |
| 60. | Head: weight (mature stage; | 536 | gr | 543 | gr |
| | average of 4-5) | | | | |

| | | | |
|---|---|---|---|
| 61. | Resistance to Bremia lactucae (Bl) | Present | Present |
| | Isolate Bl: 32 EU | | |
| 62 | Resistance to Bremia lactucae (Bl) | Present | Present |
| | Isolate Bl: 34 EU | | |
| 63. | Resistance to Bremia lactucae (Bl) | Present | Present |
| | Isolate Bl: 36 EU | | |
| 64. | Resistance to Lettuce root aphid | Absent | Absent |
| | (Pb) | | |
| 65. | Resistance to Lettuce Necrotic | Absent | Absent |
| | Stunt Virus LNSV | | |
| 66. | Resistance to Lettuce Big Vein | Absent | Absent |
| | Virus LBVV | | |
| 67. | Head: diameter of the cut of the | Medium | big |
| | trunk | | |
| 68. | Head: height of the bottom ribs | Low | Medium |

### Lettuce Variety ‘P-2019107’

Provided herein is lettuce variety ‘P-2019107,’ a crisphead lettuce variety suitable for production in the spring and winter harvesting seasons in, for example, Spain. Lettuce variety ‘P-2019107’ is the result of a cross between a mother crisphead line that was susceptible to Bremia lactucae and a Bremia lactucae-resistant crisphead line, and was selected, following backcrosses and selfing. These plants were chosen for their dark to very dark green leaf color and resistance to Bremia lactucae.

Lettuce variety ‘P-2019107’ has uniformity and stability for these traits, within the limits of environmental influence for the traits. It has been self-pollinated through a sufficient number of generations with careful attention to uniformity of plant type. The variety has been propagated with continuous attention for uniformity.

### Breeding History

Crosses were made by spraying or misting water over the flowers, thereby inactivating pollen present on these flowers. This treatment enables the cross pollination of Lactuca plants.

F1 plants can be self-pollinated to produce the F2 generation, etc.

A Bremia lactucae-susceptible crisphead line was used as the mother variety, and was crossed with a Bremia lactucae-resistant line. The Bremia lactucae-resistant line is described in U.S. Pat. No. 10,575,479 and U.S. Patent Application Publication No. 2020/0029523, both of which are incorporated by reference herein in their entirety.

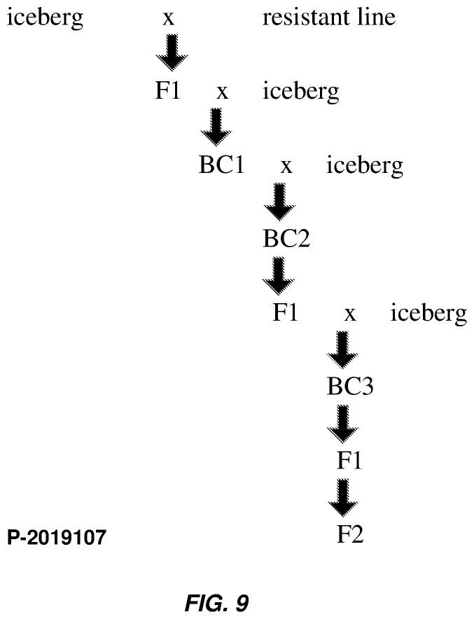

From these crosses, the F1 plants with the greatest resistance to Bremia lactucae were identified and a single plant was selected and backcrossed with the mother line. This was repeated, and the backcross line was sown and planted in the field, where the best performing line was selected. This best-performing line was backcrossed with the mother line again, the best-performing line was selected and selfed for two generations. The best-performing line was then selected (FIG. 9).

Seeds of all plants from this most-uniform line were collectively harvested and designated crisphead variety ‘P-2019107’. A deposit of seeds of ‘P-2019107’ as disclosed above and recited in the appended claims has been made with the National Collections of Industrial, Food and Marine Bacteria (NCIMB), Ferguson Building, Craibstone Estate, Bucksburn, Aberdeen AB21 9YA, United Kingdom. The date of the deposit was Feb. 7, 2020. All restrictions will be irrevocably removed upon granting of a patent, and the deposit is intended to meet all of the requirements of 37 C.F.R. § 1.801-1.809. The NCIMB Accession Number is 43572. The deposit will be maintained in the depository for a period of thirty years, or five years after the last request, or for the enforceable life of the patent, whichever is longer, and will be replaced as necessary during that period.

### Variety Description

Lettuce variety ‘P-2019107’ is a seed-propagated crisphead lettuce which is open pollinated. Variety ‘P-2019107’ is resistant to at least Bremia lactucae isolates Bl:16-36, Nasonovia ribisnigri:0, and LMV Strain LS-1. Lettuce variety ‘P-2019107’ has black seeds, exhibits dark to very dark green leaves and late bolting under long day conditions, and is comparable to the variety ‘Damice’. With regard to this comparison, variety ‘P-2019107’ exhibits a larger head, superior cold tolerance, and a later planting slot (November versus October for ‘Damice’).

Characteristics of ‘P-2019107’ in concordance with the applicable UPOV Questionnaire, as compared to the ‘Damice’ variety, are provided below in Table 9.

TABLE 9

| | Characteristic | ‘P-2019107’ | | ‘Damice’ | |
|---|---|---|---|---|---|
| 1. | Seed: color | black | | black | |
| 2. | Plant: diameter (at market stage) | 35-40 | cm | 40-45 | cm |
| 3. | Plant: degree of overlapping of | strong | | strong | |
| | upper part of leaves | | | | |
| 4. | Leaf: number of leaves | not applicable | | not applicable | |
| 5. | Leaf: attitude | semi-erect | | semi-erect | |
| 6. | Leaf: number of divisions | absent or | | absent or | |
| | | very few | | very few | |
| 7. | Leaf: shape | broad obtrullate | | broad obtrullate | |
| 8. | Leaf: shape of apex | obcordate | | obcordate | |
| 9. | Leaf: longitudinal section | convex | | convex | |
| 10. | Leaf: width or lobes [only | not applicable | | not applicable | |
| | oakleaf type] | | | | |
| 11. | Leaf: anthocyanin coloration | absent or | | absent or | |
| | | very weak | | very weak | |
| 12. | Leaf: hue of anthocyanin | not applicable | | not applicable | |
| | coloration | | | | |
| 13. | Leaf: area covered by anthocyanin | very small | | very small | |
| | coloration | | | | |
| 14. | Leaf: color | green | | green | |
| 15. | Leaf: intensity of green color | dark | | dark | |
| 16. | Leaf: glossiness of upper side | strong | | medium | |
| 17. | Leaf: thickness | thick | | thick | |
| 18. | Leaf: blistering | weak | | medium | |
| 19. | Leaf: size of blisters | medium | | medium | |
| 20. | Leaf: undulation of margin | weak | | weak | |
| 21. | Leaf: type of incisions of margin | bidendate | | irregularly | |
| | | | | dendate | |
| 22. | Leaf: depth of incisions of margin | medium | | shallow | |
| 23. | Leaf: depth of secondary incisions | shallow | | not applicable | |
| | of margin | | | | |
| 24. | Leaf: density of incisions of | sparse | | sparse | |
| | margin | | | | |
| 25. | Leaf: venation | flabellate | | flabellate | |
| 26. | Head: size | large | | large | |
| 27. | Head: shape in longitudinal | circular | | narrow oblate | |
| | section | | | | |
| 28. | Head: density | dense | | dense | |
| 29. | Stem: length | not applicable | | not applicable | |
| 30. | Stem: width | not applicable | | not applicable | |
| 31. | Stem: shape in longitudinal section | not applicable | | not applicable | |
| 32. | Stem: color | not applicable | | not applicable | |
| 33. | Stem: color of flesh | not applicable | | not applicable | |
| 34. | Time of harvest maturity | early | | early | |
| 35. | Time of beginning of bolting | late | | medium | |
| 36. | Axillary sprouting | Absent or weak | | medium | |
| 37. | Bolting stem: fasciation | absent or | | absent or | |
| | | very weak | | very weak | |
| 38. | Resistance to Bremia lactucae (Bl) | Present | | Present | |
| | Isolate Bl: 16EU | | | | |
| 39. | Resistance to Bremia lactucae (Bl) | Present | | Present | |
| | Isolate Bl: 17EU | | | | |
| 40. | Resistance to Bremia lactucae (Bl) | Present | | Present | |
| | Isolate Bl: 20EU | | | | |
| 41. | Resistance to Bremia lactucae (Bl) | Present | | Present | |
| | Isolate Bl: 21EU | | | | |
| 42. | Resistance to Bremia lactucae (Bl) | Present | | Present | |
| | Isolate Bl: 22EU | | | | |
| 43. | Resistance to Bremia lactucae (Bl) | Present | | Present | |
| | Isolate Bl: 23EU | | | | |
| 44. | Resistance to Bremia lactucae (Bl) | Present | | Present | |
| | Isolate Bl: 24EU | | | | |
| 45. | Resistance to Bremia lactucae (Bl) | Present | | Present | |
| | Isolate Bl: 25EU | | | | |
| 46. | Resistance to Bremia lactucae (Bl) | Present | | Present | |
| | Isolate Bl: 26EU | | | | |
| 47. | Resistance to Bremia lactucae (Bl) | Present | | Present | |
| | Isolate Bl: 27EU | | | | |
| 48. | Resistance to Bremia lactucae (Bl) | Present | | Present | |
| | Isolate Bl: 29EU | | | | |
| 49. | Resistance to Bremia lactucae (Bl) | Present | | Present | |
| | Isolate Bl: 30EU | | | | |
| 50. | Resistance to Bremia lactucae (Bl) | Present | | Present | |
| | Isolate Bl: 31EU | | | | |
| 51. | Resistance to Bremia lactucae (Bl) | Present | | Present | |
| | Isolate Bl: 33EU | | | | |
| 52. | Resistance to Bremia lactucae (Bl) | Present | | Present | |
| | Isolate Bl: 35EU | | | | |
| 53. | Resistance to Lettuce mosaic virus | Absent | | Absent | |
| | (LMV) Pathotype II | | | | |
| 54. | Resistance to Nasonovia ribisnigri | Present | | Absent | |
| | (Nr) | | | | |
| | Biotype Nr: 0 | | | | |
| 55. | Resistance to Fusarium | susceptible | | susceptible | |
| | oxysporum f. sp. lactucae (Fol) | | | | |
| | Race 1 | | | | |
| 56. | Head: size class | 9-10/box | | 9-10/box | |
| 57. | Head: Shape | Little flat round | | ball round | |
| 58. | Head: leaf wrap over the top of the | good | | good | |
| | head | | | | |
| 59. | Head: height head (mature stage; | 11.0 | cm | 13.0 | cm |
| | from cut till top) | | | | |
| 60. | Head: weight (mature stage; | 623 | gr | 898 | gr |
| | average of 4-5) | | | | |

| | | | |
|---|---|---|---|
| 61. | Resistance to Bremia lactucae (Bl) | Present | Present |
| | Isolate Bl: 32 EU | | |
| 62 | Resistance to Bremia lactucae (Bl) | Present | Present |
| | Isolate Bl: 34 EU | | |
| 63. | Resistance to Bremia lactucae (Bl) | Present | Present |
| | Isolate Bl: 36 EU | | |
| 64. | Resistance to Lettuce root aphid | Absent | Absent |
| | (Pb) | | |
| 65. | Resistance to Lettuce Necrotic | Absent | Absent |
| | Stunt Virus LNSV | | |
| 66. | Resistance to Lettuce Big Vein | Absent | Absent |
| | Virus LBVV | | |
| 67. | Head: diameter of the cut of the | medium | big |
| | trunk | | |
| 68. | Head: height of the bottom ribs | low | medium |

### Further Embodiments

In general, breeding goals associated with development of any plant, including lettuce, are: head architecture, weight, size, leaf color, shape, texture, flavor, earliness, slow bolting, short core, good germination, high yield, uniformity at maturity, improved shelf life, resistances against pests and physical disorders, and hybrid production.

Nowadays lettuce breeding is accelerated by application of several techniques, including tissue culture for enabling wide crosses and protoplast fusions. Further, significant progress has been made, as for many other vegetable crops, by applying molecular markers. Developing these markers has led to the development of increasingly detailed genetic maps. Genomic data and maps are available, for example, from the University of California at the Davis Lettuce Genome Resource.

These maps contain data from several sources and multiple populations and comprise Quantitative Trait Loci (QTLs), and markers linked to monogenic traits. These markers can be, e.g., RFLP, AFLP, CAPS, RAMP, SSR, microsatellites, and/or SNP markers, which all are well known for persons skilled in the art. Also sequencing of (parts of the) Lactuca genome has been established. Application of these techniques also enables MAS/MARB.

As noted above, one breeding goal in L. sativa is resistance to pests. As with many crops, lettuce can be plagued with several physiological problems, like nutrient deficiencies, but also by pests (nematodes, insects, mammals, bacteria, fungi, and/or viruses) and disease caused thereby. Specific examples include bacterial diseases caused by Xanthomonas campestris, Erwinia carotavora, and Pseudomonas cichorii, fungal diseases caused by Alternaria sonchi, Bremia lactucae, and Sclerotinia sclerotiorum, and viral diseases caused by Lettuce Mosaic Virus (LMV), Tomato spotted wilt virus (TSWV), and Lettuce Big Vein virus (LBVV). Insects, in particular Nasonovia ribisnigri (lettuce aphid), can be an important vector for spreading viral diseases.

It is a major goal for breeders to develop plant material which has resistance to many of these diseases. This has led to, among others, material, which is resistant to various strains of Bremia lactucae, such as described in International Patent Application No. WO 2014/131857, incorporated herein by reference in its entirety.

As noted above, knowledge of L. sativa genetics has allowed for extensive mapping of the genome, including 50 genes related to resistance to seven major diseases. Modern molecular techniques also make it possible to stack genes, including genes for Bremia resistance.

It is feasible to introduce traits by cis-genes from L. sativa, or transgenes from any source, encoding novel forms of disease resistance, herbicide resistance, and resistance to pests by, among other techniques, siRNA (host induced gene silencing) as well as other methods which are common to the person skilled in the art.

These methods include, for example and without limitation, electroporation, Agrobacterium-mediated transformation, particle gun transformation, polyethylene glycol (PEG)-mediated protoplast transformation, and silicon whiskers transformation. Further, novel molecular techniques such as CRISPR/CAS9 can be applied for genome editing purposes. These techniques are known to those having ordinary skill in the art.

Introducing resistance to a pest or disease, either by conventional breeding or applying techniques as described above, provides an alternative to application of chemical protectants, which is expensive and also may have a negative impact on the environment, on growers, and others who work with the crop.

### Further Embodiments—Genetic Engineering

With the advent of molecular biological techniques that have allowed the isolation and characterization of genes that encode specific protein products, such as those described above and incorporated by reference, scientists in the field of plant biology have a strong interest in engineering the genome of plants to contain and express foreign genes, or additional, or modified versions of native, or endogenous, genes (driven by different promoters) in order to alter the traits of a plant in a specific manner. Any DNA sequences, whether from a different species (i.e. transgenes) or from the same species (i.e. cis-genes), which are introduced into the genome using transformation or various breeding methods, are herein collectively referred to as “transgenes.” Over the last fifteen to twenty years, several methods for producing transgenic plants have been developed, and the present invention, in particular embodiments, also relates to transformed versions of the aforementioned varieties. Suitable genetic engineering techniques for transforming, or introducing, traits into the lettuce varieties disclosed herein include, for example and without limitation, microinjection, biolistics, electroporation, chemical poration, and transformation using vectors.

Accordingly, provided herein are methods of transforming a plant or plant part of the lettuce varieties disclosed herein, or offspring thereof, by any transformation method known to those of skill in the art. A particularly common example of plant transformation involves the construction of an expression vector that will function in plant cells. Such a vector comprises DNA comprising a gene under control of, or operatively linked to, a regulatory element (for example, a promoter). The expression vector may contain one or more operably linked gene/regulatory element combinations. The vector(s) may be in the form of a plasmid, and can be used alone or in combination with other plasmids, to provide transformed lettuce plants using transformation methods as described in U.S. Pat. No. 8,530,725, which is incorporated herein by reference in its entirety, to incorporate transgenes into the genetic material of the lettuce plant(s).

The lettuce varieties disclosed herein and offspring thereof can be genetically engineered to express various phenotypes of agronomic interest. Examples hereof are genes that confer resistance to pests and/or herbicides, that confer or contribute to a value-added trait, and that control male sterility. Examples of desirable traits for lettuce are known to those of skill in the art and are disclosed in U.S. Pat. No. 8,530,725, which is incorporated herein by reference in its entirety. Methods for transforming lettuce plants or introducing desired traits are similarly disclosed in U.S. Pat. Nos. 8,530,725 and 9,642,331, which are incorporated herein by reference in their entirety.

### Further Embodiments—Additional Breeding Methods

Also provided herein are methods for producing a lettuce plant by crossing a first parent lettuce plant with a second parent lettuce plant wherein the first or second parent lettuce plant is a lettuce variety disclosed herein. Plant breeding techniques, including crossing, thus crossing the lettuce varieties disclosed herein with another plant (including the varieties disclosed herein), or with another variety (related or unrelated to the varieties disclosed herein) are well within the skill of the ordinary artisan. Thus, any such methods, i.a., selfing, backcrosses, hybrid production, crosses to populations, and the like, using the lettuce varieties disclosed herein should be considered part of this invention. All plants produced using one or more of the lettuce varieties disclosed herein as are within the scope of the disclosure, including those developed from varieties derived from the lettuce varieties disclosed herein.

In aspects of a breeding method described herein, both first and second parent lettuce plants are one or more of the lettuce varieties disclosed herein.

In other aspects, the lettuce varieties disclosed herein can be used in crosses with other, different, lettuce plants to produce the first generation (F1) lettuce hybrid seeds and plants with superior characteristics. The lettuce varieties disclosed herein can also be used for transformation where exogenous genes are introduced and expressed. Genetic variants created either through traditional breeding methods using the lettuce varieties disclosed herein or through transformation of the lettuce varieties disclosed herein by any of a number of protocols known to those of skill in the art are intended to be within the scope of the present invention.

In one aspect, the method includes the steps of: obtaining the lettuce plant, or a part thereof, of one or more of the lettuce varieties disclosed herein, utilizing said plant or plant part as a source of breeding material, and selecting a progeny plant with molecular markers in common with one or more of the lettuce varieties disclosed herein and/or with morphological and/or physiological characteristics selected from the characteristics of the lettuce varieties disclosed herein listed in Tables 1-9. Breeding steps that may be used include pedigree breeding, backcrossing, mutation breeding, and recurrent selection. As noted above, such breeding methods are known to those of skill in the art and are disclosed in, for example, U.S. Pat. Nos. 8,530,725 and 9,642,331, which are incorporated herein by reference in their entirety. In conjunction with these steps, techniques such as RFLP-enhanced selection, genetic marker enhanced selection (for example, SSR markers), and the making of double haploids may be utilized.

In another aspect the method includes producing a population of progeny lettuce plants of one or more of the lettuce varieties disclosed herein, comprising crossing one or more of the lettuce varieties disclosed herein with another lettuce plant, thereby producing a population of lettuce plants, which, on average, derive 50% of their alleles from the lettuce varieties disclosed herein. A plant of this population may be selected and repeatedly selfed or sibbed with a lettuce plant resulting from these successive filial generations, or may be backcrossed with a recurrent parent (one or more of the lettuce varieties disclosed herein). One aspect of this invention is the lettuce produced by this method and that has obtained at least 50%, at least 60%, at least 70%, at least 80%, at least 90% or more, all subranges and percentages therebetween inclusive, of its alleles from the lettuce varieties disclosed herein. Methods for determining genetic makeup (genotyping) of a lettuce plant are disclosed in, for example and without limitation, U.S. Pat. No. 9,642,332, which is incorporated by reference herein in its entirety.

One of ordinary skill in the art of plant breeding would know how to evaluate the traits of two plant varieties to determine if there is no significant difference between the two traits expressed by those varieties. Thus, the present invention includes progeny of the lettuce varieties disclosed herein comprising a combination of at least two traits of the lettuce varieties disclosed herein selected from those listed in Tables 1-9, so that said progeny lettuce plant is not significantly different for said traits than the lettuce varieties disclosed herein, as determined at the 5% significance level when grown in the same environmental conditions. Using techniques described herein, molecular markers may be used to identify said progeny plant as progeny of one or more of the lettuce varieties disclosed herein. Mean trait values may be used to determine whether trait differences are significant, and preferably the traits are measured on plants grown under the same environmental conditions.

What is claimed is:

1. A seed of lettuce variety ‘1434365,’ ‘1434366,’ ‘1434367,’ ‘1434368,’ ‘1434369,’ ‘1434370,’ ‘1434371,’ ‘1434377,’ or ‘P-2019107,’ wherein a representative sample of seed of said variety was deposited under NCIMB Accession No. 43577, 43576, 43575, 43574, 43573, 43571, 43570, 43569, or 43572, respectively.

2. A lettuce plant, part thereof, or tissue culture produced by growing the seed of claim 1.

3. A tissue culture produced from protoplasts or cells from the plant of claim 2, wherein said cells or protoplasts are produced from a plant part selected from the group consisting of leaf, pollen, embryo, cotyledon, hypocotyl, meristematic cell root, root tip, pistil, anther, ovule, flower, shoot, stem, seed, and petiole.

4. A lettuce plant regenerated from the tissue culture of claim 3, wherein the plant has all of the morphological and physiological characteristics of variety ‘1434365,’ ‘1434366,’ ‘1434367,’ ‘1434368,’ ‘1434369,’ ‘1434370,’ ‘1434371,’ ‘1434377,’ or ‘P-2019107’.

5. A method for producing a lettuce seed comprising crossing two lettuce plants and harvesting the resultant lettuce seed, wherein at least one of the two lettuce plants is the lettuce plant of claim 2.

6. The method of claim 5, wherein one of the two lettuce plants is a lettuce plant that is unrelated to variety ‘1434365,’ ‘1434366,’ ‘1434367,’ ‘1434368,’ ‘1434369,’ ‘1434370,’ ‘1434371,’ ‘1434377,’ or ‘P-2019107’.

7. The lettuce seed produced by the method of claim 5.

8. A lettuce plant, part thereof, or tissue culture produced by growing the seed of claim 7.

9. A method of introducing a desired trait into lettuce variety ‘1434365,’ ‘1434366,’ ‘1434367,’ ‘1434368,’ ‘1434369,’ ‘1434370,’ ‘1434371,’ ‘1434377,’ or ‘P-2019107,’ comprising:
(a) crossing a lettuce plant of variety ‘1434365,’ ‘1434366,’ ‘1434367,’ ‘1434368,’ ‘1434369,’ ‘1434370,’ ‘1434371,’ ‘1434377,’ or ‘P-2019107,’ wherein a representative sample of seed was deposited under NCIMB Accession No. 43577, 43576, 43575, 43574, 43573, 43571, 43570, 43569, or 43572, respectively, with a plant of another lettuce variety that comprises a desired trait to produce progeny plants, wherein the desired trait is selected from a group consisting of male sterility, herbicide resistance, pest resistance, modified bolting, and resistance to bacterial disease, fungal disease, or viral disease;
(b) selecting one or more progeny plants that have the desired trait to produce selected progeny plants;
(c) crossing the selected progeny plants with the lettuce plant of variety ‘1434365,’ ‘1434366,’ ‘1434367,’ ‘1434368,’ ‘1434369,’ ‘1434370,’ ‘1434371,’ ‘1434377,’ or ‘P-2019107’ to produce backcross progeny plants;
(d) selecting for backcross, progeny plants that have the desired trait and all of the physiological and morphological characteristics of lettuce variety ‘1434365,’ ‘1434366,’ ‘1434367,’ ‘1434368,’ ‘1434369,’ ‘1434370,’ ‘1434371,’ ‘1434377,’ or ‘P-2019107’; and
(e) repeating steps (c) and (d) two or more times in succession to produce selected third or higher backcross progeny plants that comprise the desired trait and all of the physiological and morphological characteristics of lettuce variety ‘1434365,’ ‘1434366,’ ‘1434367,’ ‘1434368,’ ‘1434369,’ ‘1434370,’ ‘1434371,’ ‘1434377,’ or ‘P-2019107’.

10. A lettuce plant produced by the method of claim 9, wherein said lettuce plant has the desired trait.

11. The lettuce plant of claim 10, wherein the desired trait is one or more of herbicide resistance, pest resistance, and male sterility.

12. The lettuce plant of claim 10, wherein the desired trait is resistance to a pest other than Bremia lactucae.

13. A seed, plant part, or tissue culture of the plant of claim 10.

14. A method of introducing a desired trait into lettuce variety ‘1434365,’ ‘1434366,’ ‘1434367,’ ‘1434368,’ ‘1434369,’ ‘1434370,’ ‘1434371,’ ‘1434377,’ or ‘P-2019107,’ comprising introducing a gene conferring a desired trait into the plant of claim 2.

15. A lettuce plant produced by the method of claim 14, wherein said lettuce plant has the desired trait.

16. The lettuce plant of claim 15, wherein the desired trait is one or more of herbicide resistance, pest resistance, and male sterility.

17. The lettuce plant of claim 15, wherein the desired trait is resistance to a pest other than Bremia lactucae.

18. A seed, plant part, or tissue culture of the plant of claim 15.

19. A lettuce plant designated ‘1434365,’ ‘1434366,’ ‘1434367,’ ‘1434368,’ ‘1434369,’ ‘1434370,’ ‘1434371,’ ‘1434377,’ or ‘P-2019107,’ representative seeds of which having been deposited under NCIMB Accession No. 43577, 43576, 43575, 43574, 43573, 43571, 43570, 43569, or 43572, respectively, wherein said plant comprises at least resistance to Bremia lactucae isolates Bl:16-Bl:36 and resistance to Nasonovia ribisnigri biotype 0.

20. A seed, plant part, or tissue culture of the plant of claim 19.
